(19) United States
(12) Patent Application Publication
(10) Pub. No.: US 2022/0090211 A1
(43) Pub. Date: Mar. 24, 2022

(54) Sample Validation for Cancer Classification

(71) Applicant: GRAIL, Inc., Menlo Park, CA (US)

(72) Inventors: Onur Sakarya, Redwood City, CA (US); Christopher-James A.V. Yakym, Mountain View, CA (US)

(21) Appl. No.: 17/458,194

(22) Filed: Aug. 26, 2021

Related U.S. Application Data
(60) Provisional application No. 63/071,951, filed on Aug. 28, 2020.

(51) Int. Cl. C12Q 1/6886; C12Q 1/6879; G16B 40/00; G16B 30/00; G16B 20/20

(52) U.S. Cl. CPC C12Q 1/6886; C12Q 1/6879; G16B 20/20; G16B 30/00; G16B 40/00

(57) ABSTRACT

Systems and methods for validating that a DNA sample is from a test subject are disclosed. The test subject reports one or more characteristics (biological sex, ethnicity, and/or age) that may be predicted from the DNA sample. The predictions are compared to the reported characteristics to validate the DNA sample. To validate according to biological sex, the system determines a Y-chromosome signal based on counts of sequence reads for a gene specific to the Y chromosome and, similarly, an X-chromosome signal using another gene specific to the X chromosome. The biological sex is predicted based on a comparison of the two signals. To validate according to ethnicity, the system predicts ethnicity based on detected allele frequencies for SNPs specific to each chromosome. To validate according to age, the system calculates the methylation densities for age-informative CpG sites. The system utilizes trained regression models to predict the age using the methylation densities.

### REFERENCE TO RELATED APPLICATIONS

The application claims benefit of U.S. Provisional Application No. 63/071,951 filed Aug. 28, 2020, which is incorporated by reference in its entirety.

### BACKGROUND

### Field of Art

Deoxyribonucleic acid (DNA) methylation plays an important role in regulating gene expression. Aberrant DNA methylation has been implicated in many disease processes, including cancer. DNA methylation profiling using methylation sequencing (e.g., whole genome bisulfite sequencing (WGBS)) is increasingly recognized as a valuable diagnostic tool for detection, diagnosis, and/or monitoring of cancer. For example, specific patterns of differentially methylated regions and/or allele specific methylation patterns may be useful as molecular markers for non-invasive diagnostics using circulating cell-free (cf) DNA. However, there remains a need in the art for improved methods for analyzing methylation sequencing data from cell-free DNA for the detection, diagnosis, and/or monitoring of diseases, such as cancer.

### SUMMARY

Early detection of a disease state (such as cancer) in subjects is important as it allows for earlier treatment and therefore a greater chance for survival. Sequencing of DNA fragments in cell-free (cf) DNA sample can be used to identify features that can be used for disease classification. For example, in cancer assessment, cell-free DNA based features (such as presence or absence of somatic variant, methylation status, or other genetic aberrations) from a blood sample can provide insight into whether a subject may have cancer, and further insight on what type of cancer the subject may have. Towards that end, this description includes systems and methods for analyzing cell-free DNA sequencing data for determining a subject's likelihood of having a disease.

An analytics system processes a multitude of sequencing data from a plurality of samples (e.g., a plurality of cancer and non-cancer samples) to identify features that are subsequently utilized for cancer classification. With the sequencing data, the analytics system is able to train and deploy a cancer classifier for generating a cancer prediction for a test sample.

Regarding which training samples are used to train the cancer classifier, the analytics uses training samples that have already been identified and labeled as having one or a number of cancer types, as well as training samples that are from healthy individuals that are labeled as non-cancer. Each training sample includes a set of fragments. For each training sample, the analytics system generates a feature vector, for example, by assigning a score to each of the identified features. The analytics system may group the training samples into sets of one or more training samples for iterative training of the cancer classifier. The analytics system inputs each set of feature vectors into the cancer classifier and adjusts classification parameters in the cancer classifier such that a function of the cancer classifier calculates cancer predictions that accurately predict the labels of the training samples in the set based on the feature vectors and the classification parameters. After iterating the above steps through each set of training samples, the cancer classifier is sufficiently trained.

During deployment, the analytics system generates a feature vector for a test sample in a similar manner to the training samples, e.g., by assigning a score to each of a plurality of features in a feature vector for each of the test samples. Then the analytics system inputs the feature vector for the test sample into the cancer classifier which returns a cancer prediction. In one embodiment, the cancer classifier may be configured as a binary classifier to return a cancer prediction of a likelihood of having or not having cancer. In another embodiment, the cancer classifier may be configured as a multiclass classifier to return a cancer prediction with prediction values for the cancer types being categorized.

In one or more embodiments, a method is disclosed for validating that a cell-free deoxyribonucleic acid (cfDNA) sample is from a test subject, the method comprising: obtaining a test sample from a test subject, wherein a biological sex of the test subject is known to be one of biological male or biological female; obtaining the cfDNA sample from the test sample; obtaining sequence reads from the cfDNA sample; determining a first count of sequence reads for a first gene found on the Y chromosome and not found on the X chromosome; normalizing the first count; determining a Y chromosome signal for the cfDNA sample based on the normalized first count of sequence reads for the second gene; determining a biological sex for the cfDNA sample based on the Y chromosome signal; and validating that the cfDNA sample is from the test subject if the determined biological sex and the known biological sex are the same. A system is also disclosed comprising a hardware processor and a non-transitory computer-readable storage medium storing executable instructions that, when executed by the hardware processor, cause the processor to perform the method.

In one embodiment, the method further comprises: determining a second count of sequence reads for a second gene found on an X chromosome of the human genome and not found on a Y chromosome of the human genome; normalizing the second count; and determining an X chromosome signal for the cfDNA sample based on the normalized second count of sequence reads for the first gene; wherein determining the biological sex for the cfDNA sample is further based on the X chromosome signal.

In one embodiment, the first count and the second count are normalized according to a sequencing depth of the cfDNA sample.

In one embodiment, determining the biological sex of the cfDNA sample comprises comparing a threshold ratio to a ratio of the Y chromosome signal for the cfDNA sample to the X chromosome signal for the cfDNA sample.

In one embodiment, determining the biological sex of the cfDNA sample comprises applying a biological sex classifier to the X chromosome signal for the cfDNA sample and the Y chromosome signal for the cfDNA sample to predict the biological sex of the cfDNA sample, wherein the biological sex classifier is trained with a training set of training samples, each training sample has a biological sex known to be one of biological male or biological female.

In one embodiment, the method further comprises: determining a third count of sequence reads for a third gene found on the Y chromosome and not found on the X chromosome; determining a fourth count of sequence reads for a fourth gene found on the X chromosome and not found on the Y chromosome; normalizing the third count and the fourth count; wherein determining the Y chromosome signal is further based on the normalized third count; and wherein determining the X chromosome signal is further based on the normalized fourth count.

In one embodiment, the first count, the second count, the third count, and the fourth count are normalized according to a sequencing depth of the cfDNA sample.

In one embodiment, the Y chromosome signal is an average of the normalized first count and the normalized third count, and wherein the X chromosome signal is an average of the normalized second count and the normalized fourth count.

In one embodiment, determining the biological sex of the cfDNA sample comprises comparing the Y chromosome signal for the cfDNA sample to a threshold Y chromosome signal, wherein the cfDNA sample is determined to be biological male if the Y chromosome signal for the cfDNA sample is above the threshold Y chromosome signal, and wherein the cfDNA sample is determined to be biological female if the Y chromosome signal for the cfDNA sample is below the threshold Y chromosome signal.

In one embodiment, the method further comprises, responsive to validating the cfDNA sample: filtering the sequence reads with p-value filtering to generate a set of anomalous fragments; generating a test feature vector by generating, for each of a plurality of CpG sites, a score based on whether one or more anomalous fragments overlaps the CpG site; inputting the test feature vector into a trained model to generate a cancer prediction for the test sample; and determining whether the test sample is likely to have cancer according to the cancer prediction.

In one embodiment, the sequence reads comprise methylation sequencing data generated by methylation sequencing of the cfDNA fragments.

In one embodiment, the methylation sequencing comprises WGBS.

In one embodiment, the methylation sequencing comprises targeted sequencing.

In one or more embodiments, a method is disclosed for validating that a cell-free deoxyribonucleic acid (cfDNA) sample is from a test subject, the method comprising: obtaining a test sample from a test subject, wherein the test sample is reported to be one or more reported ethnicities of a plurality of ethnicities; obtaining the cfDNA sample from the test subject; obtaining a plurality of sequence reads from the cfDNA sample, the plurality of sequence reads including a plurality of single nucleotide polymorphisms (SNPs); determining from the plurality of sequence reads, an allele frequency for each of the plurality of SNPs; obtaining expected allele frequencies for each of the plurality of SNPs for each of the plurality of ethnicities determined from a training set, wherein the ethnicity is known for each training sample in the training set; for each chromosome of a plurality of chromosomes: calculating an ethnicity probability for each of the plurality of ethnicities based on the determined allele frequencies for a subset of SNPs within the chromosome and the expected allele frequencies for the plurality of ethnicities for the subset of SNPs within the chromosome; predicting one or more ethnicities for the cfDNA sample based on the calculated ethnicity probabilities for the plurality of chromosomes; and validating that the cfDNA sample is from the test subject based on the one or more predicted ethnicities of the cfDNA sample and the one or more reported ethnicities of the test subject. A system is also disclosed comprising a hardware processor and a non-transitory computer-readable storage medium storing executable instructions that, when executed by the hardware processor, cause the processor to perform the method.

In one embodiment, the method further comprises: determining a genotype for each of the plurality of SNPs based on the allele frequency at the SNP.

In one embodiment, for each chromosome of the plurality of chromosomes, calculating the ethnicity probability for each of the plurality of ethnicities is further based on the determined genotypes for the subset of SNPs within the chromosome.

In one embodiment, for each chromosome of the plurality of chromosomes, calculating the ethnicity probability for each of the plurality of ethnicities comprises calculating a Bayesian probability based on the determined genotypes for the subset of SNPs within the chromosome.

In one embodiment, the method further comprises: determining a genotype proportion of each ethnicity of the plurality of ethnicities for the determined genotype for each of the plurality of SNPs based on the expected allele frequencies for the plurality of ethnicities, wherein calculating the Bayesian probability is further based on the determined genotype proportions.

In one embodiment, the method further comprises: for each chromosome of the plurality of chromosomes, ranking the plurality of ethnicities according to the determined ethnicity probabilities, wherein a first predicted ethnicity comprises an ethnicity of the plurality of ethnicities corresponding to a largest number of the chromosomes ranking the first ethnicity first.

In one embodiment, a second predicted ethnicity comprises an ethnicity of the plurality of ethnicities corresponding to a second largest number of the chromosomes ranking the second ethnicity first.

In one embodiment, validating that the cfDNA sample is from the test subject comprises determining that at least one of the first ethnicity prediction and the second ethnicity prediction matches one of the one or more reported ethnicities.

In one embodiment, the method further comprises, responsive to validating the cfDNA sample: filtering the sequence reads with p-value filtering to generate a set of anomalous fragments; generating a test feature vector by generating, for each of a plurality of CpG sites, a score based on whether one or more anomalous fragments overlaps the CpG site; inputting the test feature vector into a trained model to generate a cancer prediction for the test sample; and determining whether the test sample is likely to have cancer according to the cancer prediction.

In one embodiment, the sequence reads comprise methylation sequencing data generated by methylation sequencing of the cfDNA fragments.

In one embodiment, the methylation sequencing comprises WGBS.

In one embodiment, the methylation sequencing comprises targeted sequencing.

In one or more embodiments, a method is disclosed for validating that a cell-free deoxyribonucleic acid (cfDNA) sample is from a test subject, the method comprising: obtaining a test sample from a test subject, wherein an age of the test subject is reported to be within one of a plurality of age ranges; receiving the cfDNA sample from the test sample; obtaining sequence reads from the cfDNA sample; for each of a plurality of CpG sites, determining a methylation density at each of the plurality of CpG sites based on the sequence reads from the cfDNA sample; predicting an age range for the cfDNA sample by applying a trained regression model to the determined methylation densities for the plurality of CpG sites, wherein the trained regression model is trained using a training set where the methylation density for each of the plurality of CpG sites and an age is known for each individual of the training set; validating that the cfDNA sample is from the test subject based on the predicted age range of the cfDNA sample and the reported age range of the test subject. A system is also disclosed comprising a hardware processor and a non-transitory computer-readable storage medium storing executable instructions that, when executed by the hardware processor, cause the processor to perform the method.

In one embodiment, the plurality of CpG sites is identified from an initial set of CpG sites found to be correlated with age, and wherein the plurality of CpG sites are identified by excluding CpG sites from the initial set of CpG sites that are confounding features for cancer prediction.

In one embodiment, the plurality of CpG sites is identified by further excluding CpG sites from the initial set of CpG sites that are confounding features for one or both of: biological sex and ethnicity.

In one embodiment, the plurality of CpG sites is identified by: training a plurality of regression models, each regression model trained with a training set of training samples and comprising a learned coefficient for each CpG site of an initial set of CpG sites, wherein a learned coefficient for a given CpG site represents a predictive power of the CpG site; for each CpG site of the initial set of CpG sites, determining an informative score calculated as an average of the learned coefficients for the CpG site over the plurality of regression models divided by a variance of the learned coefficients for the CpG site over the plurality of regression models; ranking the CpG sites of the initial set of CpG sites according to the determined informative scores; and selecting the plurality of CpG sites from the ranking.

In one embodiment, the trained regression model is trained using a linear regression operation.

In one embodiment, the trained regression model is trained using a logistic regression operation.

In one embodiment, the trained regression model is trained using a Glmnet's regression operation with regularization implementation

In one embodiment, the method further comprises, responsive to validating the cfDNA sample: filtering the sequence reads with p-value filtering to generate a set of anomalous fragments; generating a test feature vector by generating, for each of a second plurality of CpG sites, a score based on whether one or more anomalous fragments overlaps the CpG site; inputting the test feature vector into a trained model to generate a cancer prediction for the test sample; and determining whether the test sample is likely to have cancer according to the cancer prediction.

In one embodiment, the sequence reads comprise methylation sequencing data generated by methylation sequencing of the cfDNA fragments.

In one embodiment, the methylation sequencing comprises WGBS.

In one embodiment, the methylation sequencing comprises targeted sequencing.

In one embodiment, the plurality of CpG sites comprise CpG sites listed in Table A.

In one or more embodiments, a method is disclosed for validating that a cell-free deoxyribonucleic acid (cfDNA) sample is from a test subject, the method comprising: obtaining a test sample from a test subject, wherein two or more of a biological sex, an ethnicity, and an age within one of a plurality of age ranges have been reported for the test subject; obtaining the cfDNA sample from the test sample; obtaining a plurality of sequence reads from the cfDNA sample; predicting for the cfDNA sample two or more of: a biological sex for the cfDNA sample based on: a first count of sequence reads for a first gene found on an X chromosome of the human genome and not found on a Y chromosome of the human genome, and a second count of sequence reads for a second gene found on the Y chromosome and not found on the X chromosome; one or more ethnicities for the cfDNA sample based on ethnicity probabilities calculated for each chromosome of a plurality of chromosomes, the ethnicity probabilities for a given chromosome based on an allele frequency determined from the sequence reads of the cfDNA sample for each of a plurality of SNPs on the given chromosome; and an age range for the cfDNA sample based on a methylation density determined for each of a plurality of CpG sites; and validating that the cfDNA sample is from the test subject based on a comparison of two or more of the predicted biological sex of the cfDNA sample, the one or more predicted ethnicities of the cfDNA sample, the predicted age range of the cfDNA sample and two or more of the reported biological sex, the reported ethnicity, and reported age range of the test subject. A system is also disclosed comprising a hardware processor and a non-transitory computer-readable storage medium storing executable instructions that, when executed by the hardware processor, cause the processor to perform the method.

In one or more embodiments, a method is disclosed for validating that a cell-free deoxyribonucleic acid (cfDNA) sample is from a test subject, the method comprising: obtaining a test sample from a test subject, wherein a biological sex and an ethnicity have been reported for the test subject; obtaining the cfDNA sample from the test sample; obtaining a plurality of sequence reads from the cfDNA sample; predicting for the cfDNA sample: (1) a biological sex for the cfDNA sample based on: a first count of sequence reads for a first gene found on an X chromosome of the human genome and not found on a Y chromosome of the human genome, and a second count of sequence reads for a second gene found on the Y chromosome and not found on the X chromosome; and (2) one or more ethnicities for the cfDNA sample based on ethnicity probabilities calculated for each chromosome of a plurality of chromosomes, the ethnicity probabilities for a given chromosome based on an allele frequency determined from the sequence reads of the cfDNA sample for each of a plurality of SNPs on the given chromosome; and validating that the cfDNA sample is from the test subject based on a comparison of the predicted biological sex of the cfDNA sample and the one or more predicted ethnicities of the cfDNA sample to the reported biological sex and the reported ethnicity. A system is also disclosed comprising a hardware processor and a non-transitory computer-readable storage medium storing executable instructions that, when executed by the hardware processor, cause the processor to perform the method.

In one or more embodiments, a method is disclosed for validating that a cell-free deoxyribonucleic acid (cfDNA) sample is from a test subject, the method comprising: obtaining a test sample from a test subject, wherein a biological sex and an age within one of a plurality of age ranges have been reported for the test subject; obtaining the cfDNA sample from the test sample; obtaining a plurality of sequence reads from the cfDNA sample; predicting for the cfDNA sample: (1) a biological sex for the cfDNA sample based on: a first count of sequence reads for a first gene found on an X chromosome of the human genome and not found on a Y chromosome of the human genome, and a second count of sequence reads for a second gene found on the Y chromosome and not found on the X chromosome; and (2) an age range for the cfDNA sample based on a methylation density determined for each of a plurality of CpG sites; and validating that the cfDNA sample is from the test subject based on a comparison of the predicted biological sex of the cfDNA sample and the predicted age range of the cfDNA sample to the reported biological sex and the reported age range of the test subject. A system is also disclosed comprising a hardware processor and a non-transitory computer-readable storage medium storing executable instructions that, when executed by the hardware processor, cause the processor to perform the method.

In one or more embodiments, a method is disclosed for validating that a cell-free deoxyribonucleic acid (cfDNA) sample is from a test subject, the method comprising: obtaining a test sample from a test subject, wherein an ethnicity and an age within one of a plurality of age ranges have been reported for the test subject; obtaining the cfDNA sample from the test sample; obtaining a plurality of sequence reads from the cfDNA sample; predicting for the cfDNA sample: (1) one or more ethnicities for the cfDNA sample based on ethnicity probabilities calculated for each chromosome of a plurality of chromosomes, the ethnicity probabilities for a given chromosome based on an allele frequency determined from the sequence reads of the cfDNA sample for each of a plurality of SNPs on the given chromosome; and (2) an age range for the cfDNA sample based on a methylation density determined for each of a plurality of CpG sites; and validating that the cfDNA sample is from the test subject based on a comparison of the one or more predicted ethnicities of the cfDNA sample and the predicted age range of the cfDNA sample to the reported ethnicity and the reported age range of the test subject. A system is also disclosed comprising a hardware processor and a non-transitory computer-readable storage medium storing executable instructions that, when executed by the hardware processor, cause the processor to perform the method.

### BRIEF DESCRIPTION OF DRAWINGS

FIG. 1A illustrates a flowchart describing a process of sequencing a fragment of cell-free (cf) DNA to obtain a methylation state vector, according to an embodiment.

FIG. 1B is an illustration of the process of FIG. 1A of sequencing a fragment of cell-free (cf) DNA to obtain a methylation state vector, according to an embodiment.

FIG. 2 illustrates a flowchart describing a process of performing a sequencing assay to generate sequence reads, according to an embodiment.

FIG. 3 illustrates a flowchart describing a process of validating that a cfDNA sample is from a test subject, according to an embodiment.

FIG. 4 illustrates a flowchart describing a process of predicting a gender for a cfDNA sample, according to an embodiment.

FIG. 5 illustrates a flowchart describing a process of predicting an ethnicity for a cfDNA sample, according to an embodiment.

FIG. 6 illustrates a flowchart describing a process of predicting an age for a cfDNA sample, according to an embodiment.

FIGS. 7A and 7B illustrate flowcharts describing a process of determining anomalously methylated fragments from a sample, according to an embodiment.

FIG. 8A illustrates a flowchart describing a process of training a cancer classifier, according to an embodiment.

FIG. 8B illustrates an example generation of feature vectors used for training the cancer classifier, according to an embodiment.

FIG. 9A illustrates a flowchart of devices for sequencing nucleic acid samples according to an embodiment.

FIG. 9B illustrates a block diagram of an analytics system, according to an embodiment.

FIGS. 10 and 11 illustrate graphs depicting gender determination accuracy.

FIGS. 12-14 illustrate tables depicting ethnicity prediction accuracy across chromosomes.

FIGS. 15 and 16 illustrate confusion matrices depicting ethnicity prediction accuracy with different sets of ethnicities used for classification.

FIGS. 17A & 17B illustrates graphs depicting performance of features for feature selection.

FIG. 18 illustrates graphs depicting age prediction accuracy of each feature individually.

FIG. 19 illustrates a graph depicting correlation between chronological age and determined age.

FIGS. 20A & 20B illustrates a graph depicting age prediction accuracy with selected features and regularized performance.

FIG. 21 illustrates graphs comparing age prediction accuracy considering different sets of features.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

I.A. Overview of Methylation

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy individuals, may provide insight into a subject's cancer status. As is well known in the art, DNA methylation anomalies (compared to healthy controls) can cause different effects, which may contribute to cancer. Various challenges arise in the identification of anomalously methylated cfDNA fragments. First off, determining a DNA fragment to be anomalously methylated only holds weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site causally influences methylation at a subsequent CpG site. To encapsulate this dependency is another challenge in itself.

Methylation typically occurs in deoxyribonucleic acid (DNA) when a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation is characterized for a DNA fragment, if the DNA fragment comprises more than a threshold number of CpG sites with more than a threshold percentage of those CpG sites being methylated or unmethylated.

Those of skill in the art will appreciate that the principles described herein are equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein are the same, and consequently the inventive concepts described herein are applicable to those other forms of methylation.

I.B. Definitions

The term “individual” refers to a human individual. The term “healthy individual” refers to an individual presumed to not have a cancer or disease. The term “subject” refers to an individual who is known to have, or potentially has, a cancer or disease.

The term “cell free nucleic acid” or “cfNA” refers to nucleic acid fragments that circulate in an individual's body (e.g., blood) and originate from one or more healthy cells and/or from one or more cancer cells. The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “sequence read” refers to a nucleotide sequence obtained from a nucleic acid molecule from a test sample from an individual. Sequence reads can be obtained through various methods known in the art.

The term “sequencing depth” or “depth” refers to a total number of sequence reads or read segments at a given genomic location or loci from a test sample from an individual.

The term “allele frequency” refers to a percentage of sequence reads from a test sample from an individual that are of a first allele of a plurality of alleles for a genetic locus in the genome, wherein alleles for a genetic locus refers to different nucleotide sequences of the genetic locus. For a genetic locus, a reference allele refers to the nucleotide sequence of a reference genome and alternate allele refers to any nucleotide sequence that is a variant to the reference genome.

The term “anomalous fragment,” “anomalously methylated fragment,” or “fragment with an anomalous methylation pattern” refers to a fragment that has anomalous methylation of CpG sites. Anomalous methylation of a fragment may be determined using probabilistic models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “anomaly score” refers to a score for a CpG site based on a number of anomalous fragments (or, in some embodiments, UFXMs) from a sample overlaps that CpG site. The anomaly score is used in context of featurization of a sample for classification.

### II. Sample Processing

II.A. Generating Methylation State Vectors for DNA Fragments

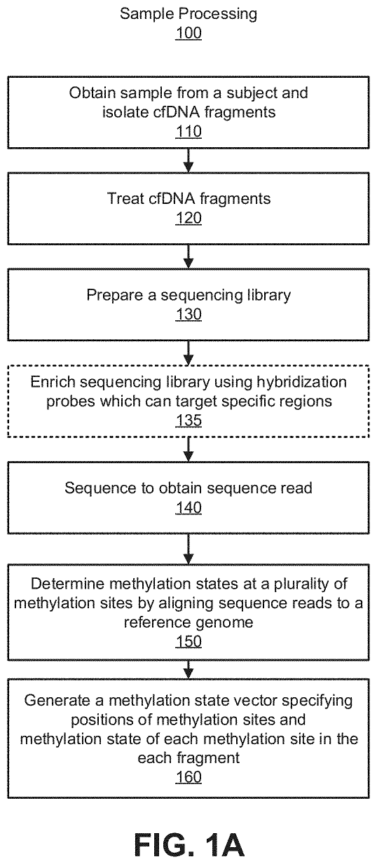

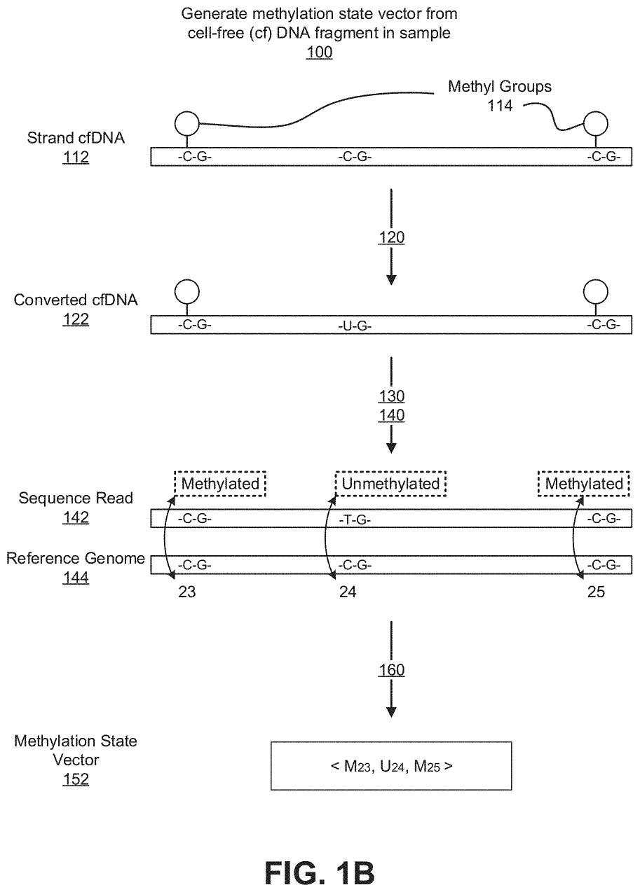

FIG. 1A is a flowchart describing a process 100 of sequencing a fragment of cell-free (cf) DNA to obtain a methylation state vector, according to an embodiment. In order to analyze DNA methylation, an analytics system first obtains 110 a test sample from an individual inclusive of at least a cfDNA sample comprising a plurality of cfDNA molecules. Generally, samples may be from healthy individuals, subjects known to have or suspected of having cancer, or subjects where no prior information is known. The test sample may be a sample selected from the group consisting of blood, plasma, serum, urine, fecal, and saliva samples. Alternatively, the test sample may comprise a sample selected from the group consisting of whole blood, a blood fraction (e.g., white blood cells (WBCs)), a tissue biopsy, pleural fluid, pericardial fluid, cerebral spinal fluid, and peritoneal fluid. In additional embodiments, the process 100 may be applied to sequence other types of DNA molecules.

From the sample, the analytics system isolates each cfDNA molecule. The cfDNA molecules are treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cfDNA molecules, a sequencing library is prepared 130. Optionally, the sequencing library may be enriched 135 for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. In one embodiment, the hybridization probes are designed to enrich for DNA molecules that have been treated (e.g., using bisulfite) for conversion of unmethylated cytosines to uracils. Once prepared, the sequencing library or a portion thereof can be sequenced to obtain a plurality of sequence reads. The sequence reads may be in a computer-readable, digital format for processing and interpretation by computer software.

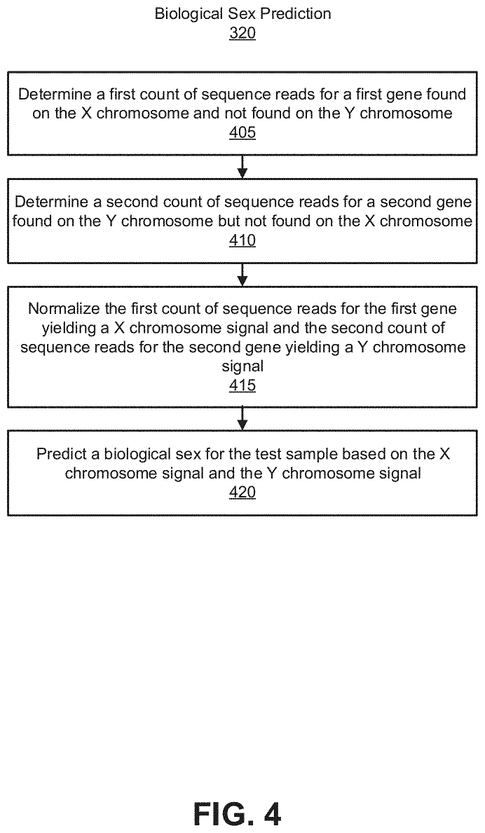

From the sequence reads, the analytics system determines 150 a location and methylation state for each CpG site based on alignment to a reference genome. The analytics system generates 160 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses; one such model will be described below in conjunction with FIG. 4.

FIG. 1B is an illustration of the process 100 of FIG. 1A of sequencing a cfDNA molecule to obtain a methylation state vector, according to an embodiment. As an example, the analytics system receives a cfDNA molecule 112 that, in this example, contains three CpG sites. As shown, the first and third CpG sites of the cfDNA molecule 112 are methylated 114. During the treatment step 120, the cfDNA molecule 112 is converted to generate a converted cfDNA molecule 122. During the treatment 120, the second CpG site which was unmethylated has its cytosine converted to uracil. However, the first and third CpG sites were not converted.

After conversion, a sequencing library 130 is prepared and sequenced 140 generating a sequence read 142. The analytics system aligns 150 the sequence read 142 to a reference genome 144. The reference genome 144 provides the context as to what position in a human genome the fragment cfDNA originates from. In this simplified example, the analytics system aligns 150 the sequence read 142 such that the three CpG sites correlate to CpG sites 23, 24, and 25 (arbitrary reference identifiers used for convenience of description). The analytics system thus generates information both on methylation status of all CpG sites on the cfDNA molecule 112 and the position in the human genome that the CpG sites map to. As shown, the CpG sites on sequence read 142 which were methylated are read as cytosines. In this example, the cytosines appear in the sequence read 142 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system generates 160 a methylation state vector 152 for the fragment cfDNA 112. In this example, the resulting methylation state vector 152 is <M₂₃, U₂₄, M₂₅>, wherein M corresponds to a methylated CpG site, U corresponds to an unmethylated CpG site, and the subscript number corresponds to a position of each CpG site in the reference genome.

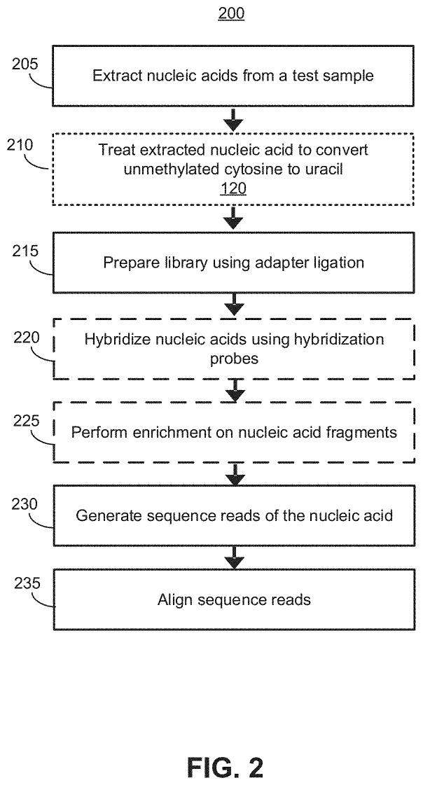

FIG. 2 illustrates a flowchart describing a process 200 of performing a sequencing assay to generate sequence reads, in accordance with an embodiment. The process 200 is a more general process flow of performing a sequencing assay compared to the process 100 which describes one embodiment of methylation sequencing. The process 200 includes, but is not limited to, the following steps. For example, any step of the process 200 may comprise a quantitation sub-step for quality control or other laboratory assay procedures known to one skilled in the art.

Generally, various sub-combinations of the steps (e.g., steps 205-235) are performed for each of the whole genome sequencing assay, small variant sequencing assay, and methylation sequencing assay. Specifically, steps 205, 215, 230, and 235 are performed for the whole genome sequencing assay. Steps 205 and 215-235 are performed for the small variant sequencing assay. In some embodiments, each of steps 205-235 are performed for the methylation sequencing assay. For example, a methylation sequencing assay that employs a targeted gene panel bisulfite sequencing employs each of steps 205-235. In some embodiments, steps 205-215 and 230-235 are performed for the methylation sequencing assay. For example, a methylation sequencing assay that employs whole genome bisulfite sequencing need not perform steps 220 and 225.

At step 205, nucleic acids (DNA or RNA) are extracted from a test sample. In the present disclosure, DNA and RNA may be used interchangeably unless otherwise indicated. That is, the following embodiments for using error source information in variant calling and quality control may be applicable to both DNA and RNA types of nucleic acid sequences. However, the examples described herein may focus on DNA for purposes of clarity and explanation. In various embodiments, DNA (e.g., cfDNA) is extracted from the test sample through a purification process. In general, any known method in the art can be used for purifying DNA. For example, nucleic acids can be isolated by pelleting and/or precipitating the nucleic acids in a tube. The extracted nucleic acids may include cfDNA or it may include gDNA, such as WBC DNA.

In step 210, the cfDNA fragments are treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA METHYLATION™—Gold, EZ DNA METHYLATION™—Direct or an EZ DNA METHYLATION™—Lightning kit (available from Zymo Research Corp, Irvine, Calif.) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

At step 215, a sequencing library is prepared. During library preparation, adapters, for example, include one or more sequencing oligonucleotides for use in subsequent cluster generation and/or sequencing (e.g., known P5 and P7 sequences for used in sequencing by synthesis (SBS) (Illumina, San Diego, Calif.)) are ligated to the ends of the nucleic acid fragments through adapter ligation. In one embodiment, unique molecular identifiers (UMI) are added to the extracted nucleic acids during adapter ligation. The UMIs are short nucleic acid sequences (e.g., 4-10 base pairs) that are added to ends of nucleic acids during adapter ligation. In some embodiments, UMIs are degenerate base pairs that serve as a unique tag that can be used to identify sequence reads obtained from nucleic acids. As described later, the UMIs can be further replicated along with the attached nucleic acids during amplification, which provides a way to identify sequence reads that originate from the same original nucleic acid segment in downstream analysis.

In step 220, hybridization probes are used to enrich a sequencing library for a selected set of nucleic acids. Hybridization probes can be designed to target and hybridize with targeted nucleic acid sequences to pull down and enrich targeted nucleic acid fragments that may be informative for the presence or absence of cancer (or disease), cancer status, or a cancer classification (e.g., cancer type or tissue of origin). In accordance with this step, a plurality of hybridization pull down probes can be used for a given target sequence or gene. The probes can range in length from about 40 to about 160 base pairs (bp), from about 60 to about 120 bp, or from about 70 bp to about 100 bp. In one embodiment, the probes cover overlapping portions of the target region or gene. For targeted gene panel sequencing, the hybridization probes are designed to target and pull down nucleic acid fragments that derive from specific gene sequences that are included in the targeted gene panel. For whole exome sequencing, the hybridization probes are designed to target and pull down nucleic acid fragments that derive from exon sequences in a reference genome. As one of skill in the art would readily appreciate, other known means in the art for targeted enrichment of nucleic acids may be used.

After a hybridization step 220, the hybridized nucleic acid fragments are enriched 225. For example, the hybridized nucleic acid fragments can be captured and amplified using PCR. The target sequences can be enriched to obtain enriched sequences that can be subsequently sequenced. This improves the sequencing depth of sequence reads.

In step 230, the nucleic acids are sequenced to generate sequence reads. Sequence reads may be acquired by known means in the art. For example, a number of techniques and platforms obtain sequence reads directly from millions of individual nucleic acid (e.g., DNA such as cfDNA or gDNA) molecules in parallel. Such techniques can be suitable for performing any of targeted gene panel sequencing, whole exome sequencing, whole genome sequencing, targeted gene panel bisulfite sequencing, and whole genome bisulfite sequencing.

As a first example, sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye.

In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain.

Any suitable sequencing-by-synthesis platform can be used to identify mutations. Sequencing-by-synthesis platforms include the Genome Sequencers from Roche/454 Life Sciences, the GENOME ANALYZER from Illumina/SOLEXA, the SOLID system from Applied BioSystems, and the HELISCOPE system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair) can be linked to each fragment to be captured on a surface coated with a respective second member of that coupling pair. Subsequent to the capture, the sequence can be analyzed, for example, by single molecule detection/sequencing, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Massively parallel sequencing or next generation sequencing (NGS) techniques include synthesis technology, pyrosequencing, ion semiconductor technology, single-molecule real-time sequencing, sequencing by ligation, nanopore sequencing, or paired-end sequencing. Examples of massively parallel sequencing platforms are the Illumina HISEQ or MISEQ, ION PERSONAL GENOME MACHINE, the PACBIO RSII sequencer or SEQUEL System, Qiagen's GENEREADER, and the Oxford MINION. Additional similar current massively parallel sequencing technologies can be used, as well as future generations of these technologies.

At step 230, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments (e.g., in paired-end sequencing), a sequence read is comprised of a read pair denoted as R_1 and R_2. For example, the first read R_1 may be sequenced from a first end of a nucleic acid fragment whereas the second read R_2 may be sequenced from the second end of the nucleic acid fragment. Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. An output file having SAM (sequence alignment map) format or BAM (binary alignment map) format may be generated and output for further analysis.

Following step 235, the aligned sequence reads are processed using a computational analysis, such as computational analysis 140B, 140C, or 140D as described above and shown in FIG. 1D. Each of the small variant computational analysis 140C, whole genome computation assay 140B, methylation computational analysis 140D, and baseline computational analysis are described in further detail below.

II.B. Sample Swap Validation

The analytics system validates that a cfDNA sample obtained from a test sample for a test subject is indeed from the test subject. The analytics system validates the cfDNA sample by predicting one or more characteristics of the test subject based on the cfDNA sample and comparing the predicted characteristics against one or more reported characteristics from the test subject. These characteristics may include but are not limited to biological sex (or gender), ethnicity, age, some other genetic trait, some other physical trait, or any combination thereof. More generally, the analytics system may validate that the test sample is indeed from the test subject by predicting the one or more characteristics based on the cfDNA molecules and/or other nucleic acid molecules present in the test sample, e.g., gDNA. As such, it should be noted that the principles discussed may reference interchangeably a test sample and a cfDNA sample obtained from the test sample.

Validating that the cfDNA sample (or more generally the test sample) is indeed from the test subject aims to reduce sample swap errors. Sample swap errors may occur at numerous junctures from collection of the test sample from the test subject to just prior to performing a sequencing assay. For example, Sample A is listed as having been collected by Test Subject A, but may truly have originated from Test Subject B, the error due to a mislabeling by a clinician. One example validation evaluates whether the biological sex predicted for Sample A matches the reported biological sex of Test Subject A. If the predicted biological sex of the sample matches the reported biological sex, then the analytics system validates the sample. Conversely, if the predicted biological sex does not match the reported biological sex, then the analytic system invalidates the sample. Invalidated samples, i.e., samples determined to not have originated from the test subject, may be excluded from any further analysis by the analytics system. The analytics system may request collection of a new sample from the test subject, e.g., through a healthcare provider. Validation of test samples consequently prevents reporting conclusions to test subjects that are derived from incorrect (e.g., swapped) test samples.

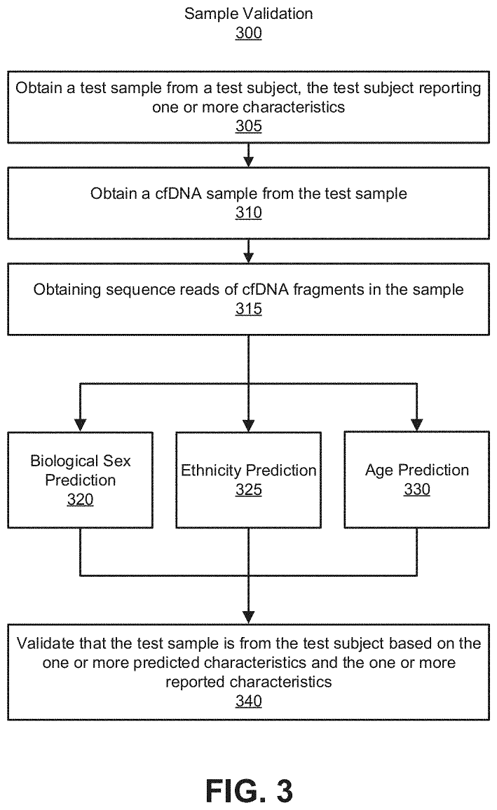

FIG. 3 illustrates a flowchart describing a process 300 of validating that a cfDNA sample is from a test subject, according to an embodiment. The analytics system may more generally validate whether the entire test sample is from the test subject with the process 300. The process 300 is described as being performed by the analytics system; however, in other embodiments, other systems and/or devices may perform one or more of the steps listed in the process 300.

The analytics system obtains 305 a test sample from a test subject, the test subject reporting one or more characteristics. The test sample includes at least the cfDNA sample and may further comprise other nucleic acid molecules. The test sample may be collected by a healthcare provider (e.g., a nurse, a physician, a clinician, etc.) or self-collected by the test subject. The test subject may report these characteristics to the healthcare provider, via a survey, via another appropriate method, etc.

The analytics system obtains 310 a cfDNA sample from the test sample. The cfDNA sample comprises a plurality of cfDNA fragments. In other embodiments, other nucleic acid molecules may also be obtained and used in subsequent steps of the process 300.

The analytics system obtains 315 sequence reads of the cfDNA fragments in the cfDNA sample. The sequence reads may be obtained via the process 100 in FIG. 1A and/or the process 200 in FIG. 2. In some embodiments, the analytics system further obtains a methylation state vector for each of the cfDNA fragments from the sequence reads, e.g., via the process 100 in FIG. 1A.

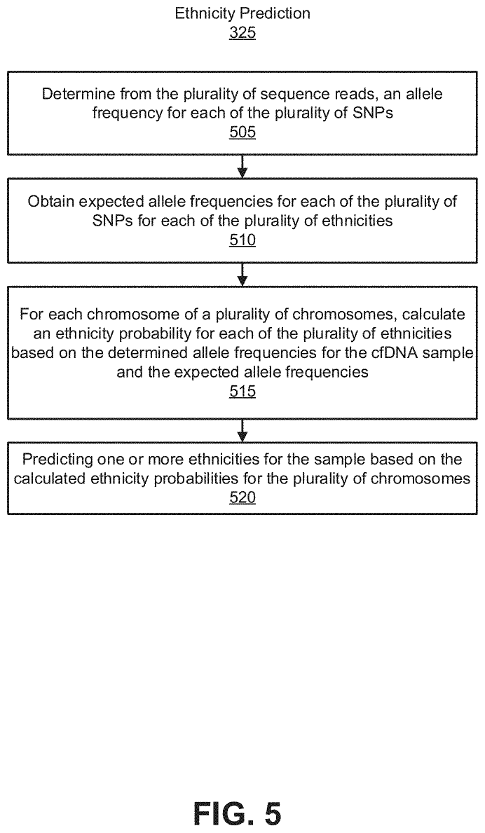

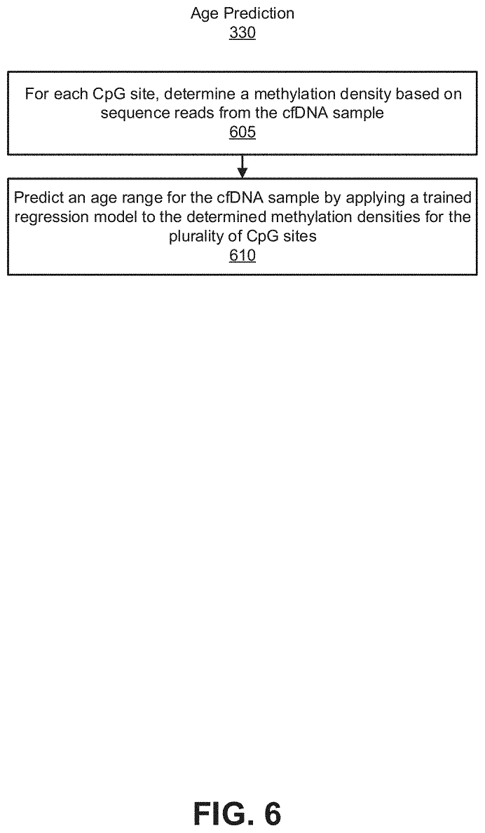

The analytics system predicts one or more characteristics of the cfDNA sample. In the embodiment shown in FIG. 3, the analytics system performs a biological sex prediction 320 yielding a predicted biological sex for the test sample, an ethnicity prediction 325 yielding at least one predicted ethnicity for the test sample, an age prediction 330 yielding a predicted age range for the test sample, or some combination thereof In other embodiments, the analytics system predicted additional characteristics. The biological sex prediction 320 is further described in FIG. 4. The ethnicity prediction 325 is further described in FIG. 5. The age prediction 330 is further described in FIG. 6.

The analytic system validates 340 that the test sample is from the test subject based on the one or more predicted characteristics and the one or more reported characteristics. For each characteristic evaluated in the validation, the analytics system predicts whether the predicted characteristic matches the reported characteristic.

For biological sex, the analytics system determines whether the reported biological sex characteristic matches the predicted biological sex characteristic. For example, if the test subject reported a biological sex characteristic of female, then the analytics system evaluates whether the predicted biological sex characteristic is also female, which would match the reported characteristic. Similarly, if the test reported a biological sex characteristic of male, then the analytics system evaluates whether the predicted biological sex characteristic is also male, which would match the reported characteristic.

For ethnicity, the analytics system determines whether the reported one or more ethnicity characteristics match the predicted one or more ethnicity characteristics. For test subjects that reported a single ethnicity, the analytics system determines whether a first ranked prediction matches the reported ethnicity. As an example, a test subject reported an ethnicity characteristic of African, then the analytics system evaluates whether the predicted ethnicity characteristic is also African, which matches the reported characteristic. In some embodiments, the analytics system provides a second ranked prediction in addition to the first ranked prediction. In these embodiments, the analytics system reports a match if either the first ranked prediction or the second ranked prediction matches the reported ethnicity. For subjects that may be of mixed ethnicity (i.e., of two or more ethnicities) reporting two or more ethnicities, then the analytics system may evaluate whether the first ranked prediction and the second ranked prediction (or subsequent prediction(s)) match at least two of the ethnicities reported.

For age, the analytics system determines whether the reported age range (inclusive of the test subject's age) matches the predicted age range. As an example, if the test subject reported an age characteristic of 35 (or an age characteristic of an age range inclusive of the test subject's age), then the analytics system evaluates whether the predicted age range (e.g., the age range of 30-40) is inclusive of the age of 35 (or matches the reported age range), which would match the reported characteristic.

In one embodiment, all characteristics evaluated need to match in order for the test sample to be validated. For example, when evaluating age and biological sex, the predicted age range must match the reported age and the predicted biological sex must match the reported biological sex in order for the cfDNA sample to be validated as belonging to the test subject. In other embodiments, a majority consensus between the various characteristics suffices to validate the cfDNA sample as originating from the test subject. For example, when evaluating age, biological sex, and ethnicity, at least two of the three characteristics need to be satisfied in order to validate that the cfDNA sample is from the test subject.

II.B.I. Biological Sex Prediction

FIG. 4 illustrates a flowchart describing a process of biological sex prediction 320 for a cfDNA sample, according to an embodiment. Biological sex refers to which sex chromosomes an individual has in their genome. The majority of individuals have either a biological sex of two X chromosomes (“biological female”) or a biological sex of one X chromosome and one Y chromosome (“biological male”). There are some individuals with sex chromosomal abnormalities which deviate from the majority. These sex chromosomal abnormalities include Klinefelter Syndrome with an individual having two X chromosomes and one Y chromosome (categorized as biological male), Turner Syndrome with an individual having one X chromosome and one missing or partial X chromosome (categorized as biological female), Trisomy X with an individual having three X chromosomes (categorized as biological female), Tetrasomy X with an individual having four X chromosomes (categorized as biological female). It should be noted that a test subject may be asked to provide a gender from which a biological sex may be deduced. The process of biological sex prediction 320 is described as being performed by the analytics system; however, in other embodiments, other systems and/or devices may perform one or more of the steps listed in the process 320.

The analytics system determines 405 a first count of sequence reads for a first gene found on an X chromosome in the cfDNA sample for the test subject and not found on a Y chromosome (such a gene found on the X chromosome and not the Y chromosome may be referred to as a X-specific gene). Each sequence read may be aligned to the human genome such that the analytics system may determine that each sequence read overlaps which genes. The analytics system identifies the sequence reads inclusive of the first gene and counts the first count of the identified sequence reads. In some embodiments, the analytics system determines a third count of sequence reads for a third gene also found on an X chromosome and not found on a Y chromosome to corroborate the first count.

The analytics system determines 410 a second count of sequence reads for a second gene found on a Y chromosome in the cfDNA sample for the test subject and not found on an X chromosome (such a gene found on the Y chromosome and not the X chromosome may be referred to as a Y-specific gene). The analytics system identifies the sequence reads inclusive of the second gene and counts the second count of the identified sequence reads. In some embodiments, the analytics system determines a fourth count of sequence reads for a fourth gene also found on a Y chromosome and not found on an X chromosome to corroborate the second count.

The analytics system normalizes 415 the first count of sequence reads for the first gene yielding a X chromosome signal and the second count of sequence reads for the second gene yielding a Y chromosome signal. The analytics system may normalize according to the sequencing depth of the cfDNA sample. The resulting normalized first count is the X chromosome signal in the cfDNA sample, and the normalized second count is the Y chromosome signal in the cfDNA sample. In embodiments with the third count of the third gene that was X-specific and the fourth count that was Y-specific, the analytics system may similarly normalize the third count and the fourth count. The average between the first count and the third count can be used as the X chromosome signal. Likewise, the average between the second count and the fourth count can be used as the Y chromosome signal. The analytics system may extend these principles to factor any number of X-specific genes to derive the X chromosome signal and any number of Y-specific genes to derive the Y chromosome signal.

In one embodiment, the analytics system predicts 420 a biological sex for the test sample based on the Y chromosome signal. The analytics system determines and applies a threshold Y chromosome signal to determine between biological male and biological female. Test samples having Y chromosome signals at or above the threshold Y chromosome signal are determined to be biological male, and test samples having Y chromosome signals below the threshold Y chromosome signal are determined to be biological female. Using a threshold Y chromosome signal works as no biological female should have significant Y chromosome signal. The threshold Y chromosome signal may be determined using a set of training samples with some training samples that are biological male and other training samples that are biological female. The analytics system sequences each training sample to obtain sequence reads (e.g., via the process 100 or the process 200), and performs steps 405, 410, and 415 of the process 320. The analytics system plots the training samples according to X chromosome signal and Y chromosome signal. The analytics system may then identify the threshold Y chromosome signal that captures all the biological males in the set of training samples. In some embodiments, the analytics system predicts the biological sex further based on the X chromosome signal. The analytics system may identify (via a similar process described to identify the threshold Y chromosome signal) a threshold X chromosome signal. The analytics system may predict the biological sex for a test sample using a combination of the threshold X chromosome signal and the threshold Y chromosome signal.

In another embodiment, the analytics system calculates a ratio between the X chromosome signal and the Y chromosome signal. A threshold ratio may be used to determine between biological male and biological female. Similar to determining the threshold Y chromosome signal, the analytics system may use a set of training samples with some training samples that are biological male and other training samples that are biological female. The analytics system calculates an X chromosome signal and a Y chromosome signal for each training sample. The analytics system may then determine the threshold ratio that accurately classifies between biological male and biological female for the training samples.

In other embodiments, the analytics system applies a trained biological sex classifier to the X chromosome signal and the Y chromosome signal. The analytics system trains the biological sex classifier using a set of training samples with some training samples that are biological male and other training samples that are biological female. The analytics system calculates an X chromosome signal and a Y chromosome signal for each training sample. The analytics system trains the biological sex classifier by inputting the training samples and adjusting weights of the biological sex classifier to accurately predict the known biological sex of the training samples. Neural networks and other machine learning algorithms may be implemented in training the biological sex classifier.

II.B.II. Ethnicity Prediction

FIG. 5 illustrates a flowchart describing a process of ethnicity prediction 325 for a cfDNA sample, according to an embodiment. The test subject may report being of one or more ethnicities from a plurality of ethnicities. The process of ethnicity prediction 325 is described as being performed by the analytics system; however, in other embodiments, other systems and/or devices may perform one or more of the steps listed in the process 325. The sequence reads obtained for the cfDNA sample cover a plurality of single nucleotide polymorphisms (SNPs).

The analytics system determines 505 from the plurality of sequence reads, an allele frequency for each of the plurality of SNPs. The plurality of SNPs may be common SNPs from the 1000 Genomes Project (also referred to as “1000G project”). A common SNP has read depth of at least 15 and has a Minor Allele Frequency (MAF) greater than or equal to 1%. The analytics system determines the allele frequency of a reference allele for the SNP by counting a percentage of the sequence reads covering that SNP which have the reference allele. The analytics system may further determine a genotype for each SNP from the allele frequency. For example: if the allele frequency of the reference allele is approximately 0, then the genotype may be determined to homozygous alternate; if the allele frequency of the reference allele is approximately 0.5, then the genotype may be determined to heterozygous; and if the allele frequency of the reference allele is approximately 1, then the genotype may be determined to be homozygous reference.

The analytics system obtains 510 expected allele frequencies for each of the plurality of SNPs for each of the plurality of ethnicities. The analytics system obtains a training set of individuals with sequence reads derived from a cfDNA sample, e.g., according to process 100 of FIG. 1 of process 200 of FIG. 2. The individuals have one or more known ethnicities, by which ethnicity cohorts may be established. In some embodiments, only individuals that report one ethnicity are used in the training set such that individuals are not of mixed ethnicity. The analytics system, for each ethnicity and for each SNP, determines an expected allele frequency. For M ethnicities and N SNPs considered, this yields M times N expected allele frequencies. In one or more embodiments, the training set is derived from an external database.

The analytics system may determine a percentage of each genotype for the SNP from the expected allele frequencies. The proportion of a population at equilibrium belonging to each genotype can be calculated via the Hardy-Weinberg equation. The Hardy-Weinberg equation is expressed as:

$${p}^{2} + 2 pq + {q}^{2} = 1 \qquad ( 1 )$$

In Equation (1): p refers to one allele frequency (e.g., the reference allele frequency), and q refers to the other allele frequency (e.g., the alternate allele frequency). The percentage of each genotype is broken down such that homozygous reference is the term p², heterozygous is the term 2pq, and homozygous alternate is the term q².

The analytic system, for each chromosome of a plurality of chromosomes, calculates 515 an ethnicity probability for each of the plurality of ethnicities based on the determine allele frequencies for the cfDNA sample and the expected allele frequencies. In one embodiment, the analytics system calculates the ethnicity probability for an ethnicity given the determined allele frequencies for the plurality of SNPs on each chromosome as a Bayesian probability derived from the Bayes rule, which can be expressed as:

$$P ( {E}_{x} \vert D ) = \frac{P ( D \vert {E}_{x} ) * P ( {E}_{x} )}{P ( D )} \qquad ( 2 )$$

In Equation 2: P(Eₓ|D) is the ethnicity probability for ethnicity x represented as Eₓ given the genotypes D over the SNPs N on a chromosome determined based on the allele frequencies for the cfDNA sample; the right side of Equation 2 represents the Bayesian probability of P(Eₓ|D); P(D|Eₓ) is the probability that someone of ethnicity Eₓ has the genotypes D over the SNPs on the chromosome that match the cfDNA sample; P(Eₓ) is the probability of being ethnicity Eₓ; and P(D) is the probability of observing the genotypes D over the SNPs. The terms on the righthand side of Equation 2 can be approximated with the expected allele frequencies of the training set, serving as a representative sample of the global population. As such, P(D|Eₓ) can be calculated as follows:

$$P ( D \vert {E}_{x} ) = {\prod }_{i = 1}^{N} P ( {D}_{i} \vert {E}_{x} ) \qquad ( 3 )$$

In Equation 3: P(D|Eₓ) is calculated as a product operator over the probability of the genotype Dᵢ of the cfDNA sample over all the SNPs N on the chromosome in the ethnicity Eₓ cohort of the training set. The term P(Dᵢ|Eₓ) can be calculated via the Hardy-Weinberg equation, Equation 1, with the expected allele frequencies of ethnicity Eₓ cohort at SNP i. P(Eₓ) is simply the proportion of the training set that belongs to the ethnicity Eₓ cohort. P(D) is calculated as follows:

$$P ( D ) = {\sum }_{j = 1}^{M} P ( {E}_{j} ) * P ( D \vert {E}_{j} ) = {\sum }_{j = 1}^{M} P ( {E}_{j} ) {\prod }_{i = 1}^{N} P ( {D}_{i} \vert {E}_{j} ) \qquad ( 4 )$$

In Equation 4: P(D) is analogous to sum operator over all ethnicities M taking the proportion of the training set that belongs to each ethnicity cohort j iterated from 1 to M multiplied by P(D|Eⱼ) which is calculated via Equation 3.

As a result of the above calculations, each of the plurality of chromosomes (under consideration) of the cfDNA sample has an ethnicity probability for each ethnicity. As an example, with 22 autosomal chromosomes being considered and 5 ethnicities classified against (East Asian, South Asian, European, Admixed American, African), Chromosome 1 has an East Asian ethnicity probability, a South Asian ethnicity probability, a European ethnicity probability, an Admixed American ethnicity probability, and an African ethnicity probability.

The analytics system predicts 520 one or more ethnicities for the cfDNA sample based on the calculated ethnicity probabilities for the plurality of chromosomes. The analytics system may rank the ethnicities for each chromosome based on the ethnicity probabilities for the chromosome. Following the example in the paragraph above, the analytics system ranks the 5 ethnicities according to ethnicity probabilities for Chromosome 1. With all the chromosomes having a rank of ethnicities, the analytics system may predict the cfDNA sample to be of the ethnicity having the majority rank of 1 across all chromosomes. For example, if the East Asian ethnicity ranked first across 20 out of 22 chromosomes considered, then the analytics system predicts the cfDNA sample to be of East Asian ethnicity (also referred to as a “first prediction”). In situations with a tie between two or more ethnicities, the analytics system may predict the cfDNA sample to be of the ethnicities that tied.

In some embodiments, the analytics system includes a second prediction (also referred to as a “second predicted ethnicity”). As one example of such embodiments, the analytics system includes a second prediction if there is not a unanimous consensus of first ranked prediction across all chromosomes considered. The second prediction is identified from dissenting chromosomes having a different first ranked prediction. In other words, the first predicted ethnicity corresponds to a largest number of the chromosomes ranking the first predicted ethnicity as first and the second predicted ethnicity corresponds to a second largest number of the chromosomes ranking the second predicted ethnicity as first. For example, 16 chromosomes ranked European as first and 6 chromosomes ranked African as first. The analytics system would return European as the first prediction given the majority agreement (16 out of 22) and African as the second prediction given the minority dissent of the majority agreement (6 out of 22). Utilizing a second prediction aids in ensuring cfDNA samples of mixed ethnicities are not falsely invalidated. In additional embodiments, second ranked predictions across chromosomes may also be considered. The analytics system may further include subsequent predictions based on a next largest number of the chromosomes ranking the subsequent predicted ethnicities as first, e.g., a third predicted ethnicity, a fourth predicted ethnicity, and so on.

II.B.III. Age Prediction

FIG. 6 illustrates a flowchart describing a process of age prediction 330 for a cfDNA sample, according to an embodiment. The test subject may report being within an age range of a plurality of age ranges. For example, age ranges can be partitioned by 10 years at a time, such that age ranges are 0-10 years, 10-20 years, 20-30 years, 30-40 years, 40-50 years, 50-60 years, 60-70 years, 70-80 years, etc. The process of age prediction 330 is described as being performed by the analytics system; however, in other embodiments, other systems and/or devices may perform one or more of the steps listed in the process 330. Age prediction 330 relies on methylation sequencing data of the cfDNA sample.

The analytics system selects a set of CpG sites as features for predicting age according to the process 330. In one embodiment, the analytics system retrieves information from an external system indicating CpG sites determined to have methylation densities correlated with age. This may serve as an initial set of CpG sites. The analytics system excludes CpG sites that are confounding features for cancer prediction (e.g., features identified according to principles described below in Section III.B. Training of Cancer Classifier). The analytics system may also control for biological sex, ethnicity, other characteristics, alcohol consumption, smoking habits, other behavioral habits, etc. The remaining CpG sites that are not confounded with cancer prediction or other characteristics are selectively used as features in regressing for age prediction.

In some embodiments, the analytics system further reduces the set of features to select some of the more informative CpG sites. For each CpG site in an initial set of CpG sites, the analytics system may repeatedly train some number of regression models with different training sets of training samples. From the regression models, the analytics system may rank CpG sites according to the learned coefficients associated with the CpG sites. A learned coefficient represents a predictive power of the CpG site. A larger learned coefficient represents a greater change in methylation density over age representing high predictive power. Alternatively, a small learned coefficient represents little to no change in methylation density over age representing low predictive power. A positive learned coefficient represents a positive correlation between methylation density and age, i.e., methylation density increases as age increases. A negative learned coefficient represents a negative correlation between methylation density and age, i.e., methylation density decreases as age increases. In some embodiments, the analytics system calculates an informative score for each CpG site according to an absolute mean of learned coefficients for the CpG site over the plurality of trained regression models divided by a variance of the learned coefficients for the CpG site. A top number of CpG sites may be selected from the ranking as the features used for predicting age.

The analytics system, for each CpG site (e.g., features selected according to the paragraph above), determines 605 a methylation density based on the sequence reads from the cfDNA sample. In some embodiments, the analytics system determines a methylation state vector from each sequence read, e.g., according to the process 100 of FIG. 1A. The methylation state vector describes a plurality of CpG sites that are covered by a particular cfDNA fragment. The methylation state vector includes a methylation state at each covered CpG site. The analytics system determines a methylation density for each CpG site by calculating a percentage of methylation state vectors (representing cfDNA fragments in the cfDNA sample) that have a methylation state of methylated. In some embodiments, only methylation state vectors have a state of methylated or unmethylated are counted while excluding methylation state vectors having a state of indeterminate.

The analytics system predicts 610 an age range for the cfDNA sample by applying a trained regression model to the determined methylation densities for the plurality of CpG sites. The trained regression model inputs the determined methylation densities for the plurality of CpG sites and outputs a predicted age range out of a plurality of age ranges. The trained regression model is trained with a training set of cfDNA samples, each cfDNA sample having known methylation densities at the plurality of CpG sites and a known age. In one or more embodiments, a regularization factor is implemented in the loss function when training the regression model. The analytics system may minimize coefficients of the loss function to model the training set. In some embodiments, optimization algorithms such as cyclical coordinate descent, gradient descent, Newton's method, Quasi-Newton methods, simplex algorithm, or other descent algorithms may be used to minimize the loss function. The analytic system may further cross-validate the trained regression model to measure the model's predictive accuracy.

II.C. Identifying Anomalous Fragments

The analytics system determines anomalous fragments for a sample using the sample's methylation state vectors. For each fragment in a sample, the analytics system determines whether the fragment is an anomalous fragment using the methylation state vector corresponding to the fragment. In one embodiment, the analytics system calculates a p-value score for each methylation state vector describing a probability of observing that methylation state vector or other methylation state vectors even less probable in the healthy control group. The process for calculating a p-value score will be further discussed below in Section II.B.i. P-Value Filtering. The analytics system may determine fragments with a methylation state vector having below a threshold p-value score as anomalous fragments. In another embodiment, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

II.C.I. P-Value Filtering

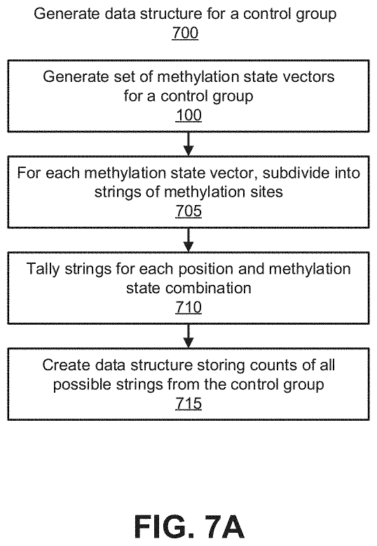

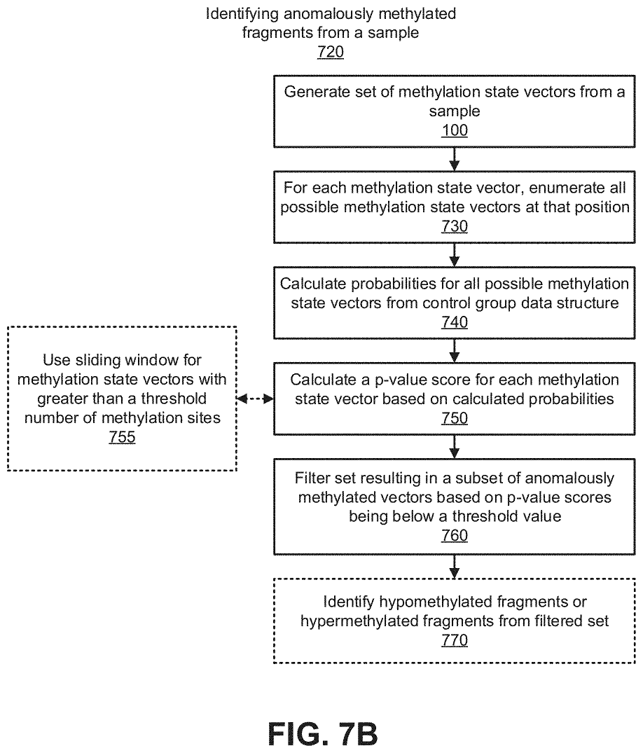

In one embodiment, the analytics system calculates a p-value score for each methylation state vector compared to methylation state vectors from fragments in a healthy control group. The p-value score describes a probability of observing the methylation status matching that methylation state vector or other methylation state vectors even less probable in the healthy control group. In order to determine a DNA fragment to be anomalously methylated, the analytics system uses a healthy control group with a majority of fragments that are normally methylated. When conducting this probabilistic analysis for determining anomalous fragments, the determination holds weight in comparison with the group of control subjects that make up the healthy control group. To ensure robustness in the healthy control group, the analytics system may select some threshold number of healthy individuals to source samples including DNA fragments. FIG. 7A below describes the method of generating a data structure for a healthy control group with which the analytics system may calculate p-value scores. FIG. 7B describes the method of calculating a p-value score with the generated data structure.

FIG. 7A is a flowchart describing a process 700 of generating a data structure for a healthy control group, according to an embodiment. To create a healthy control group data structure, the analytics system receives a plurality of DNA fragments (e.g., cfDNA) from a plurality of healthy individuals. A methylation state vector is identified for each fragment, for example via the process 100.

With each fragment's methylation state vector, the analytics system subdivides 705 the methylation state vector into strings of CpG sites. In one embodiment, the analytics system subdivides 705 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 710 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 710 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 715 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size helps keep the data structure creation and performance (e.g., use for later accessing as described below), in terms of computational and storage, reasonable. Second, a statistical consideration to limiting the maximum string length is to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it requires a significant amount of data that may not be available, and thus would be too sparse for a model to perform appropriately. For example, calculating a probability of anomalousness/cancer conditioned on the prior 100 CpG sites would require counts of strings in the data structure of length 100, ideally some matching exactly the prior 100 methylation states. If only sparse counts of strings of length 100 are available, there will be insufficient data to determine whether a given string of length of 100 in a test sample is anomalous or not.

FIG. 7B is a flowchart describing a process 720 for identifying anomalously methylated fragments from an individual, according to an embodiment. In process 720, the analytics system generates 100 methylation state vectors from cfDNA fragments of the subject. The analytics system handles each methylation state vector as follows.

For a given methylation state vector, the analytics system enumerates 730 all possibilities of methylation state vectors having the same starting CpG site and same length (i.e., set of CpG sites) in the methylation state vector. As each methylation state is generally either methylated or unmethylated there are effectively two possible states at each CpG site, and thus the count of distinct possibilities of methylation state vectors depends on a power of 2, such that a methylation state vector of length n would be associated with 2ⁿ possibilities of methylation state vectors. With methylation state vectors inclusive of indeterminate states for one or more CpG sites, the analytics system may enumerate 730 possibilities of methylation state vectors considering only CpG sites that have observed states.

The analytics system calculates 740 the probability of observing each possibility of methylation state vector for the identified starting CpG site and methylation state vector length by accessing the healthy control group data structure. In one embodiment, calculating the probability of observing a given possibility uses a Markov chain probability to model the joint probability calculation. In other embodiments, calculation methods other than Markov chain probabilities are used to determine the probability of observing each possibility of methylation state vector.

The analytics system calculates 750 a p-value score for the methylation state vector using the calculated probabilities for each possibility. In one embodiment, this includes identifying the calculated probability corresponding to the possibility that matches the methylation state vector in question. Specifically, this is the possibility of having the same set of CpG sites, or similarly the same starting CpG site and length as the methylation state vector. The analytics system sums the calculated probabilities of any possibilities having probabilities less than or equal to the identified probability to generate the p-value score.

This p-value represents the probability of observing the methylation state vector of the fragment or other methylation state vectors even less probable in the healthy control group. A low p-value score, thereby, generally corresponds to a methylation state vector which is rare in a healthy individual, and which causes the fragment to be labeled anomalously methylated, relative to the healthy control group. A high p-value score generally relates to a methylation state vector is expected to be present, in a relative sense, in a healthy individual. If the healthy control group is a non-cancerous group, for example, a low p-value indicates that the fragment is anomalous methylated relative to the non-cancer group, and therefore possibly indicative of the presence of cancer in the test subject.

As above, the analytics system calculates p-value scores for each of a plurality of methylation state vectors, each representing a cfDNA fragment in the test sample. To identify which of the fragments are anomalously methylated, the analytics system may filter 760 the set of methylation state vectors based on their p-value scores. In one embodiment, filtering is performed by comparing the p-values scores against a threshold and keeping only those fragments below the threshold. This threshold p-value score could be on the order of 0.1, 0.01, 0.001, 0.0001, or similar.

According to example results from the process 400, the analytics system yields a median (range) of 2,800 (1,500-12,000) fragments with anomalous methylation patterns for participants without cancer in training, and a median (range) of 3,000 (1,200-220,000) fragments with anomalous methylation patterns for participants with cancer in training. These filtered sets of fragments with anomalous methylation patterns may be used for the downstream analyses as described below in Section III.

In one embodiment, the analytics system uses 755 a sliding window to determine possibilities of methylation state vectors and calculate p-values. Rather than enumerating possibilities and calculating p-values for entire methylation state vectors, the analytics system enumerates possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window identifies the sequential set of CpG sites from the vector within the window starting from the first CpG site in the vector. The analytic system calculates a p-value score for the window including the first CpG site. The analytics system then “slides” the window to the second CpG site in the vector, and calculates another p-value score for the second window. Thus, for a window size/and methylation vector length m, each methylation state vector will generate m−l+1 p-value scores. After completing the p-value calculations for each portion of the vector, the lowest p-value score from all sliding windows is taken as the overall p-value score for the methylation state vector. In another embodiment, the analytics system aggregates the p-value scores for the methylation state vectors to generate an overall p-value score.

Using the sliding window helps to reduce the number of enumerated possibilities of methylation state vectors and their corresponding probability calculations that would otherwise need to be performed. To give a realistic example, it is possible for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations enumerates 2\hat{ }5 (32) possibilities of methylation state vectors, which total results in 50×2\hat{ }5 (1.6×10\hat{ }3) probability calculations. This results in a vast reduction of calculations to be performed, with no meaningful hit to the accurate identification of anomalous fragments.

In embodiments with indeterminate states, the analytics system may calculate a p-value score summing out CpG sites with indeterminates states in a fragment's methylation state vector. The analytics system identifies all possibilities that have consensus with the all methylation states of the methylation state vector excluding the indeterminate states. The analytics system may assign the probability to the methylation state vector as a sum of the probabilities of the identified possibilities. As an example, the analytics system calculates a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states uses calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In one embodiment, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities allows for efficient calculation of p-score values without needing to re-calculate the underlying probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

II.C.II. Hypermethylated Fragments and Hypomethylated Fragments

In another embodiment, the analytics system determines anomalous fragments as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

II.D. Example Analytics System

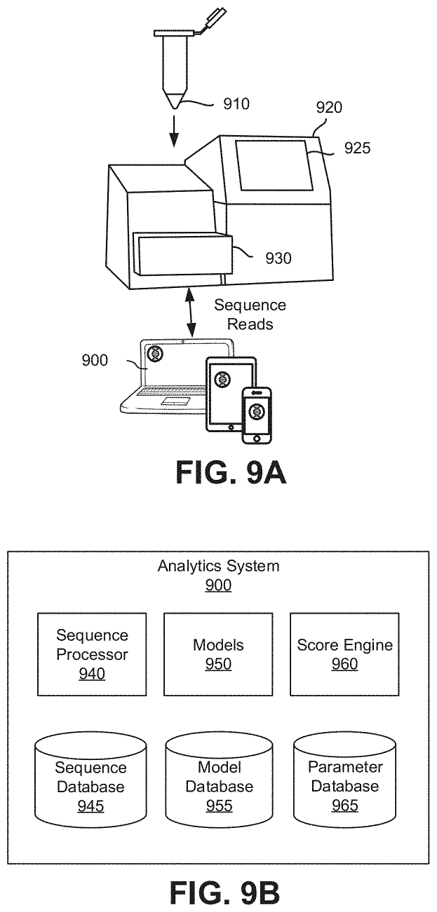

FIG. 9A is a flowchart of devices for sequencing nucleic acid samples according to one embodiment. This illustrative flowchart includes devices such as a sequencer 920 and an analytics system 900. The sequencer 920 and the analytics system 900 may work in tandem to perform one or more steps in the processeses 100 of FIG. 1A, 700 of FIG. 7A, 720 of FIG. 7B, and other process described herein.

In various embodiments, the sequencer 920 receives an enriched nucleic acid sample 910. As shown in FIG. 9A, the sequencer 920 can include a graphical user interface 925 that enables user interactions with particular tasks (e.g., initiate sequencing or terminate sequencing) as well as one more loading stations 930 for loading a sequencing cartridge including the enriched fragment samples and/or for loading necessary buffers for performing the sequencing assays. Therefore, once a user of the sequencer 920 has provided the necessary reagents and sequencing cartridges to the loading station 930 of the sequencer 920, the user can initiate sequencing by interacting with the graphical user interface 925 of the sequencer 920. Once initiated, the sequencer 920 performs the sequencing and outputs the sequence reads of the enriched fragments from the nucleic acid sample 910.

In some embodiments, the sequencer 920 is communicatively coupled with the analytics system 900. The analytics system 900 includes some number of computing devices used for processing the sequence reads for various applications such as assessing methylation status at one or more CpG sites, variant calling or quality control. The sequencer 920 may provide the sequence reads in a BAM file format to the analytics system 900. The analytics system 900 can be communicatively coupled to the sequencer 920 through a wireless, wired, or a combination of wireless and wired communication technologies. Generally, the analytics system 900 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information, e.g., via step 140 of the process 100 in FIG. 1A. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 900 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from the first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. An output file having SAM (sequence alignment map) format or BAM (binary) format may be generated and output for further analysis.

Referring now to FIG. 9B, FIG. 9B is a block diagram of an analytics system 900 for processing DNA samples according to one embodiment. The analytics system implements one or more computing devices for use in analyzing DNA samples. The analytics system 900 includes a sequence processor 940, sequence database 945, model database 955, models 950, parameter database 965, and score engine 960. In some embodiments, the analytics system 900 performs some or all of the processes 100 of FIG. 1A and 700 of FIG. 7.

The sequence processor 940 generates methylation state vectors for fragments from a sample. At each CpG site on a fragment, the sequence processor 940 generates a methylation state vector for each fragment specifying a location of the fragment in the reference genome, a number of CpG sites in the fragment, and the methylation state of each CpG site in the fragment whether methylated, unmethylated, or indeterminate via the process 100 of FIG. 1A. The sequence processor 940 may store methylation state vectors for fragments in the sequence database 945. Data in the sequence database 945 may be organized such that the methylation state vectors from a sample are associated to one another.

Further, multiple different models 950 may be stored in the model database 955 or retrieved for use with test samples. In one example, a model is a trained cancer classifier for determining a cancer prediction for a test sample using a feature vector derived from anomalous fragments. The training and use of the cancer classifier will be further discussed in conjunction with Section III. Cancer Classifier for Determining Cancer. The analytics system 900 may train the one or more models 950 and store various trained parameters in the parameter database 965. The analytics system 900 stores the models 950 along with functions in the model database 955.

During inference, the score engine 960 uses the one or more models 950 to return outputs. The score engine 960 accesses the models 950 in the model database 955 along with trained parameters from the parameter database 965. According to each model, the score engine receives an appropriate input for the model and calculates an output based on the received input, the parameters, and a function of each model relating the input and the output. In some use cases, the score engine 960 further calculates metrics correlating to a confidence in the calculated outputs from the model. In other use cases, the score engine 960 calculates other intermediary values for use in the model.

### III. Cancer Classifier for Determining Cancer

III.A. Overview

The cancer classifier is trained to receive a feature vector for a test sample and determine whether the test sample is from a test subject that has cancer or, more specifically, a particular cancer type. The cancer classifier comprises a plurality of classification parameters and a function representing a relation between the feature vector as input and the cancer prediction as output determined by the function operating on the input feature vector with the classification parameters. In one embodiment, the feature vectors input into the cancer classifier are based on a set of anomalous fragments determined from the test sample. The anomalous fragments may be determined via the process 720 in FIG. 7B, or more specifically hypermethylated and hypomethylated fragments as determined via the step 770 of the process 720, or anomalous fragments determined according to some other process. Prior to deployment of the cancer classifier, the analytics system trains the cancer classifier.

III.B. Training of Cancer Classifier

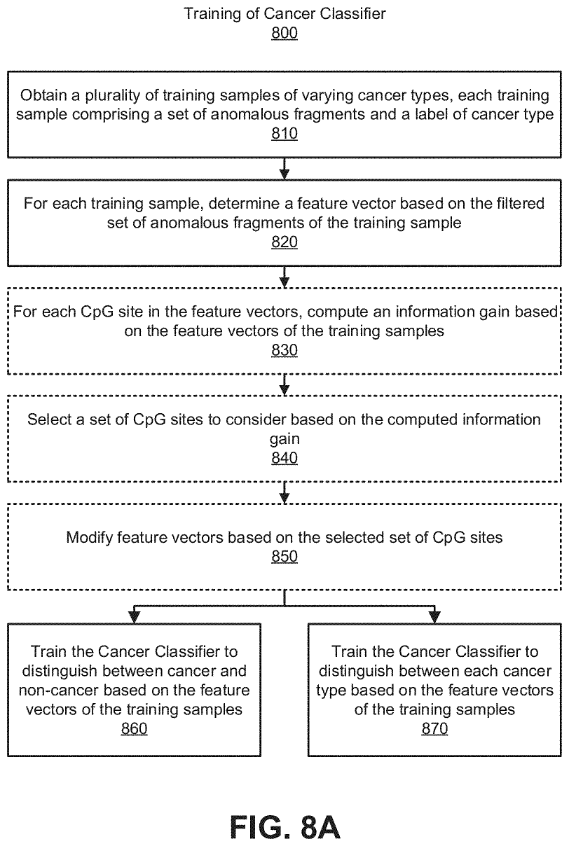

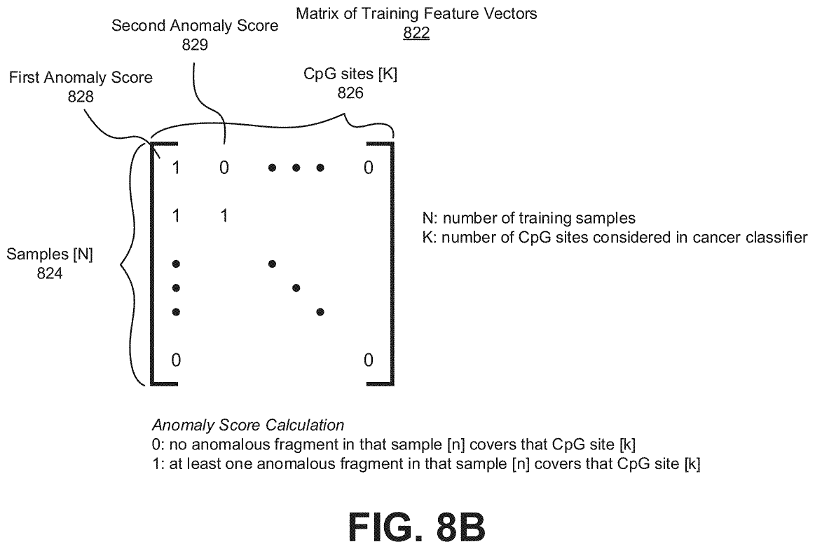

FIG. 8A is a flowchart describing a process 800 of training a cancer classifier, according to an embodiment. The analytics system obtains 810 a plurality of training samples each having a set of anomalous fragments and a label of a cancer type. The plurality of training samples includes any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 820, for each training sample, a feature vector based on the set of anomalous fragments of the training sample. The analytics system calculates an anomaly score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragments in a sample that overlap the CpG site and calculates an anomaly score based on the count of 5.

Once all anomaly scores are determined for a training sample, the analytics system determines the feature vector as a vector of elements including, for each element, one of the anomaly scores associated with one of the CpG sites in an initial set. The analytics system normalizes the anomaly scores of the feature vector based on a coverage of the sample. Here, coverage refers to a median or average sequencing depth over all CpG sites covered by the initial set of CpG sites used in the classifier, or based on the set of anomalous fragments for a given training sample.

As an example, reference is now made to FIG. 8B illustrating a matrix of training feature vectors 822. In this example, the analytics system has identified CpG sites [K] 826 for consideration in generating feature vectors for the cancer classifier. The analytics system selects training samples [N] 824. The analytics system determines a first anomaly score 828 for a first arbitrary CpG site [k1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 828 for the first CpG site as 1, as illustrated in FIG. 8B. Considering a second arbitrary CpG site [k2], the analytics system similarly checks the set of anomalous fragments for at least one that includes the second CpG site [k2]. If the analytics system does not find any such anomalous fragment that includes the second CpG site, the analytics system determines a second anomaly score 829 for the second CpG site [k2] to be 0, as illustrated in FIG. 8B. Once the analytics system determines all the anomaly scores for the initial set of CpG sites, the analytics system determines the feature vector for the first training sample [n1] including the anomaly scores with the feature vector including the first anomaly score 828 of 1 for the first CpG site [k1] and the second anomaly score 829 of 0 for the second CpG site [k2] and subsequent anomaly scores, thus forming a feature vector [1, 0, . . . ].

The analytics system may further limit the CpG sites considered for use in the cancer classifier. The analytics system computes 830, for each CpG site in the initial set of CpG sites, an information gain based on the feature vectors of the training samples. From step 820, each training sample has a feature vector that may contain an anomaly score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 830 an information gain for each cancer type and for each CpG site in the initial set to determine whether to include that CpG site in the classifier. The information gain is computed for training samples with a given cancer type compared to all other samples. For example, two random variables ‘anomalous fragment’ (‘AF’) and ‘cancer type’ (‘CT’) are used. In one embodiment, AF is a binary variable indicating whether there is an anomalous fragment overlapping a given CpG site in a given sample as determined for the anomaly score/feature vector above. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site.

For a given cancer type, the analytics system uses this information to rank CpG sites based on how cancer specific they are. This procedure is repeated for all cancer types under consideration. If a particular region is commonly anomalously methylated in training samples of a given cancer but not in training samples of other cancer types or in healthy training samples, then CpG sites overlapped by those anomalous fragments will tend to have high information gains for the given cancer type. The ranked CpG sites for each cancer type are greedily added (selected) 840 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 850 the feature vectors of the training samples as needed. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 820 or to the selected set of CpG sites from step 850. In one embodiment, the analytics system trains 860 a binary cancer classifier to distinguish between cancer and non-cancer based on the feature vectors of the training samples. In this manner, the analytics system uses training samples that include both non-cancer samples from healthy individuals and cancer samples from subjects. Each training sample has one of the two labels “cancer” or “non-cancer.” In this embodiment, the classifier outputs a cancer prediction indicating the likelihood of the presence or absence of cancer.

In another embodiment, the analytics system trains 850 a multiclass cancer classifier to distinguish between many cancer types (also referred to as tissue of origin (TOO) labels). Cancer types include one or more cancers and may include a non-cancer type (may also include any additional other diseases or genetic disorders, etc.). To do so, the analytics system uses the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier is sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

III.C. Deployment of Cancer Classifier

During use of the cancer classifier, the analytics system obtains a test sample from a subject of unknown cancer type. The analytics system may process the test sample comprised of DNA molecules with any combination of the processes 100, 700, and 720 to achieve a set of anomalous fragments. The analytics system determines a test feature vector for use by the cancer classifier according to similar principles discussed in the process 800. The analytics system calculates an anomaly score for each CpG site in a plurality of CpG sites in use by the cancer classifier. For example, the cancer classifier receives as input feature vectors inclusive of anomaly scores for 1,000 selected CpG sites. The analytics system thus determines a test feature vector inclusive of anomaly scores for the 1,000 selected CpG sites based on the set of anomalous fragments. The analytics system calculates the anomaly scores in the same manner as the training samples. In one embodiment, the analytics system defines the anomaly score as a binary score based on whether there is a hypermethylated or hypomethylated fragment in the set of anomalous fragments that encompasses the CpG site.

The analytics system then inputs the test feature vector into the cancer classifier. The function of the cancer classifier then generates a cancer prediction based on the classification parameters trained in the process 800 and the test feature vector. In the first manner, the cancer prediction is binary and selected from a group consisting of “cancer” or “non-cancer;” in the second manner, the cancer prediction is selected from a group of many cancer types and “non-cancer.” In additional embodiments, the cancer prediction has prediction values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 860 of the process 800 with another cancer classifier trained in step 870 or the process 800. The analytics system inputs the test feature vector into the cancer classifier trained as a binary classifier in step 860 of the process 800. The analytics system receives an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier receives the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system determines a cancer score for a test sample based on the test sample's sequencing data (e.g., methylation sequencing data, SNP sequencing data, other DNA sequencing data, RNA sequencing data, etc.). The analytics system compares the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

### IV. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

IV.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

IV.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction , then the treatment is considered to have been successful. However, if the second cancer prediction increases compared to the first cancer prediction , then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

IV.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V. Example Results of Cancer Classifier

V.A. Sample Collection and Processing

Study design and samples: CCGA (NCT02889978) is a prospective, multi-center, case-control, observational study with longitudinal follow-up. De-identified biospecimens were collected from approximately 15,000 participants from 142 sites. Samples were divided into training (1,785) and test (1,015) sets; samples were selected to ensure a prespecified distribution of cancer types and non-cancers across sites in each cohort, and cancer and non-cancer samples were frequency age-matched by gender.

Whole-genome bisulfite sequencing: cfDNA was isolated from plasma, and whole-genome bisulfite sequencing (WGBS; 30× depth) was employed for analysis of cfDNA. cfDNA was extracted from two tubes of plasma (up to a combined volume of 10 ml) per patient using a modified QIAamp Circulating Nucleic Acid kit (Qiagen; Germantown, Md.). Up to 75 ng of plasma cfDNA was subjected to bisulfite conversion using the EZ-96 DNA Methylation Kit (Zymo Research, D5003). Converted cfDNA was used to prepare dual indexed sequencing libraries using Accel-NGS Methyl-Seq DNA library preparation kits (Swift BioSciences; Ann Arbor, Mich.) and constructed libraries were quantified using KAPA Library Quantification Kit for Illumina Platforms (Kapa Biosystems; Wilmington, Mass.). Four libraries along with 10% PhiX v3 library (Illumina, FC-110-3001) were pooled and clustered on an Illumina NovaSeq 6000 S2 flow cell followed by 150-bp paired-end sequencing (30×).

For each sample, the WGBS fragment set was reduced to a small subset of fragments having an anomalous methylation pattern. Additionally, hyper or hypomethylated cfDNA fragments were selected. cfDNA fragments selected for having an anomalous methylation pattern and being hyper or hypermethylated, i.e., UFXM. Fragments occurring at high frequency in individuals without cancer, or that have unstable methylation, are unlikely to produce highly discriminatory features for classification of cancer status. We therefore produced a statistical model and a data structure of typical fragments using an independent reference set of 108 non-smoking participants without cancer (age: 58±14 years, 79 [73%] women) (i.e., a reference genome) from the CCGA study. These samples were used to train a Markov-chain model (order 3) estimating the likelihood of a given sequence of CpG methylation statuses within a fragment as described above in Section II.B. This model was demonstrated to be calibrated within the normal fragment range (p-value>0.001) and was used to reject fragments with a p-value from the Markov model as >=0.001 as insufficiently unusual.

As described above, further data reduction step selected only fragments with at least 5 CpGs covered, and average methylation either >0.9 (hyper methylated) or <0.1 (hypo-methylated). This procedure resulted in a median (range) of 2,800 (1,500-12,000) UFXM fragments for participants without cancer in training, and a median (range) of 3,000 (1,200-220,000) UFXM fragments for participants with cancer in training. As this data reduction procedure only used reference set data, this stage was only required to be applied to each sample once.

V.B. Sample Swap Validation

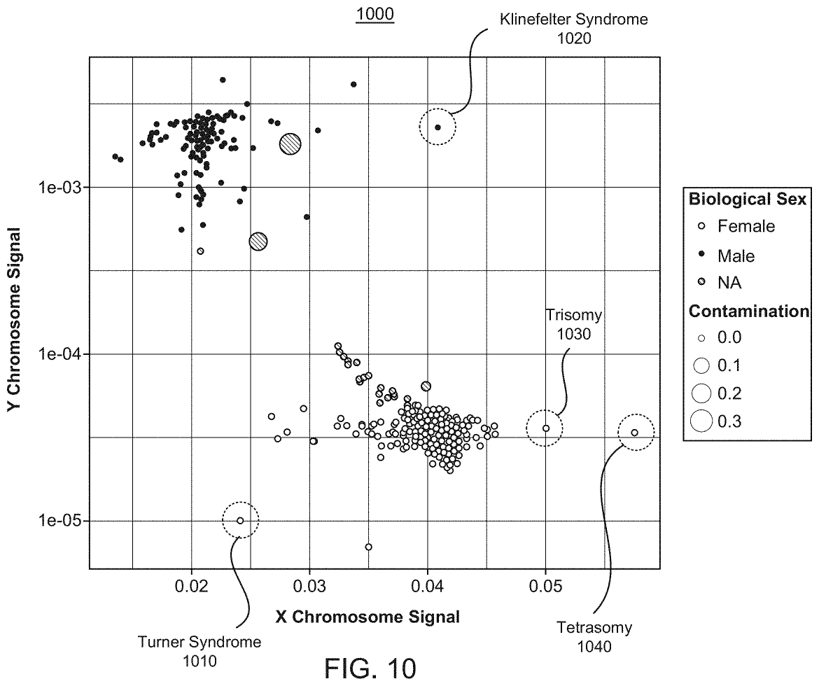

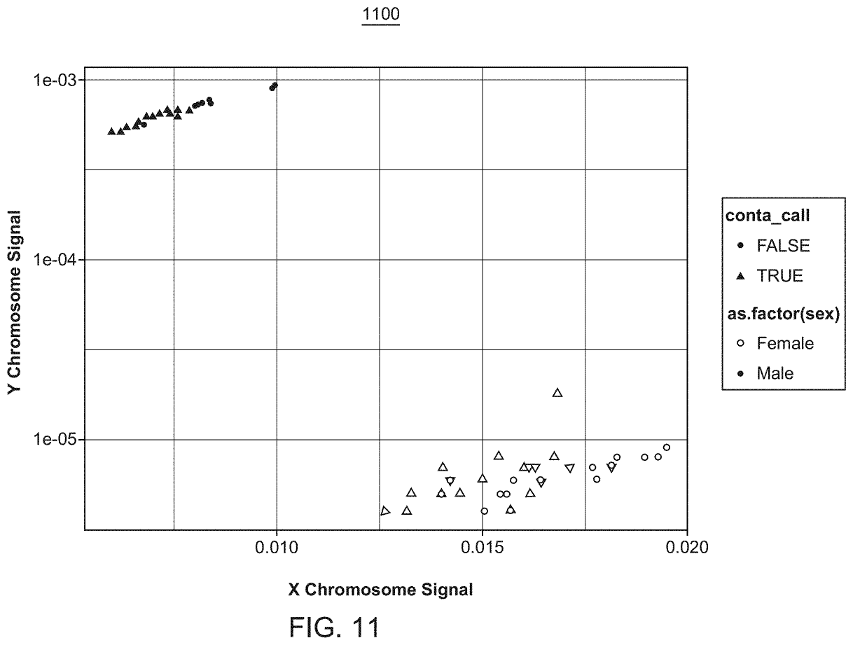

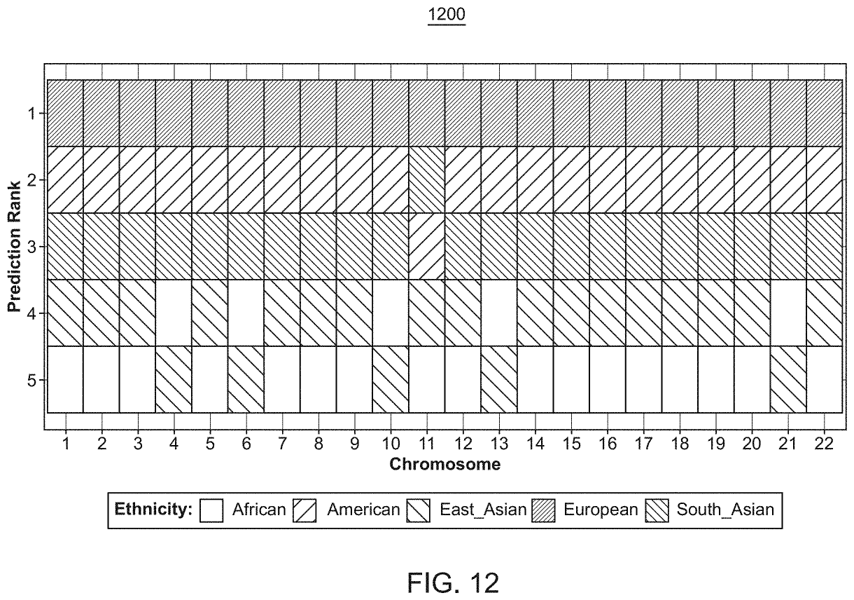

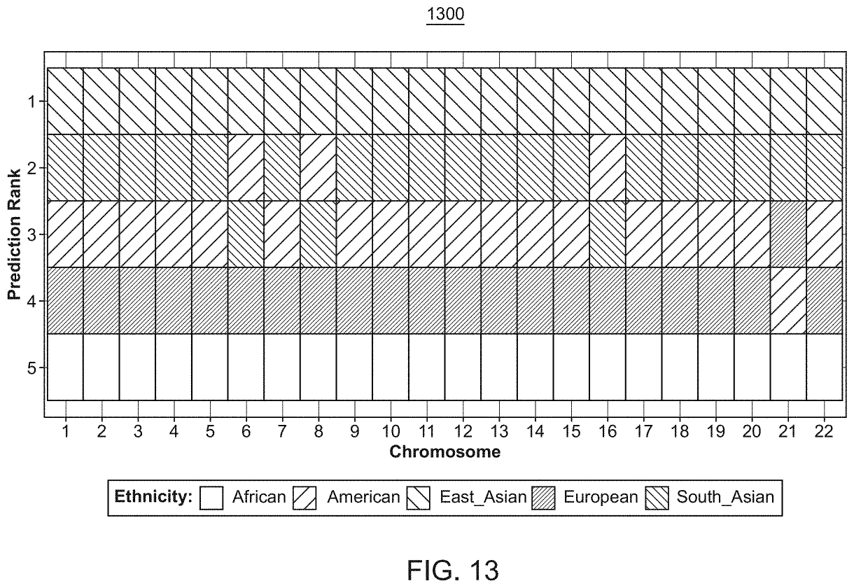

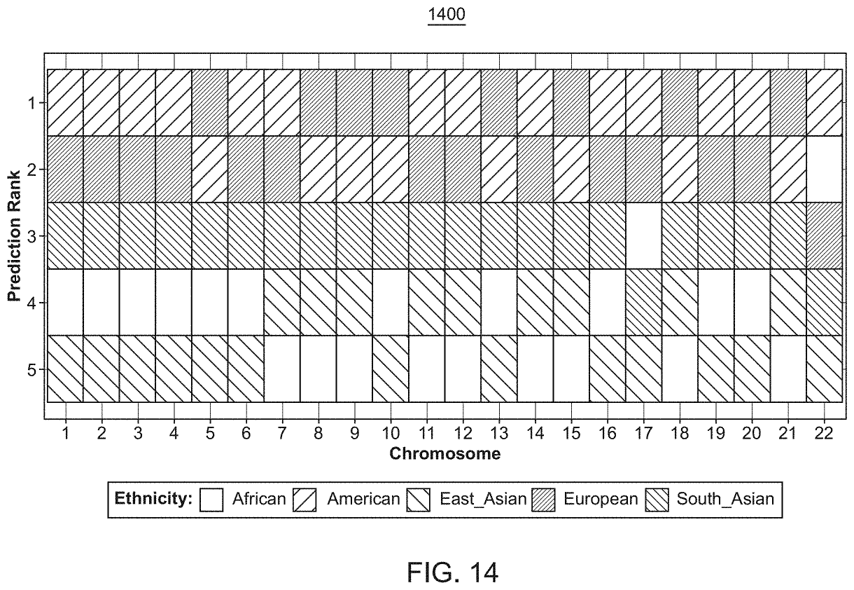

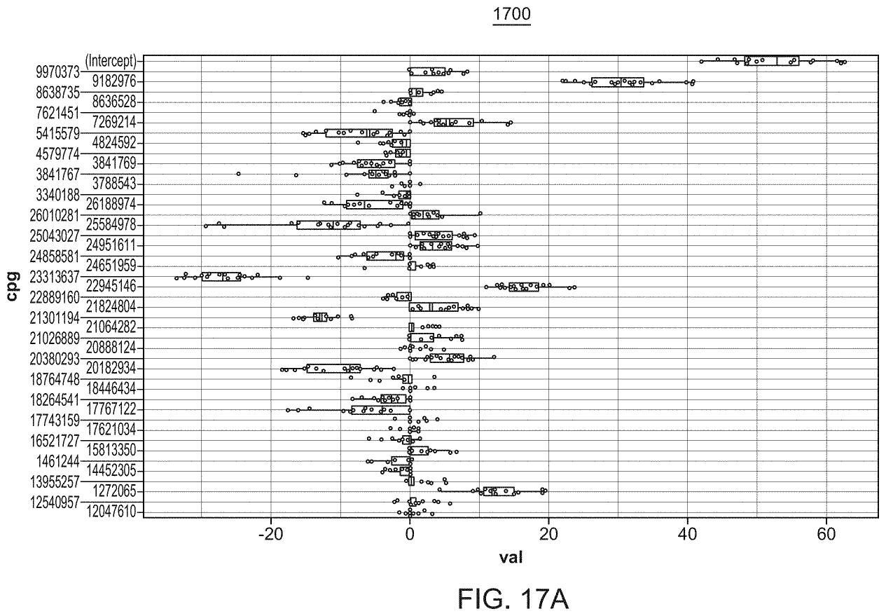

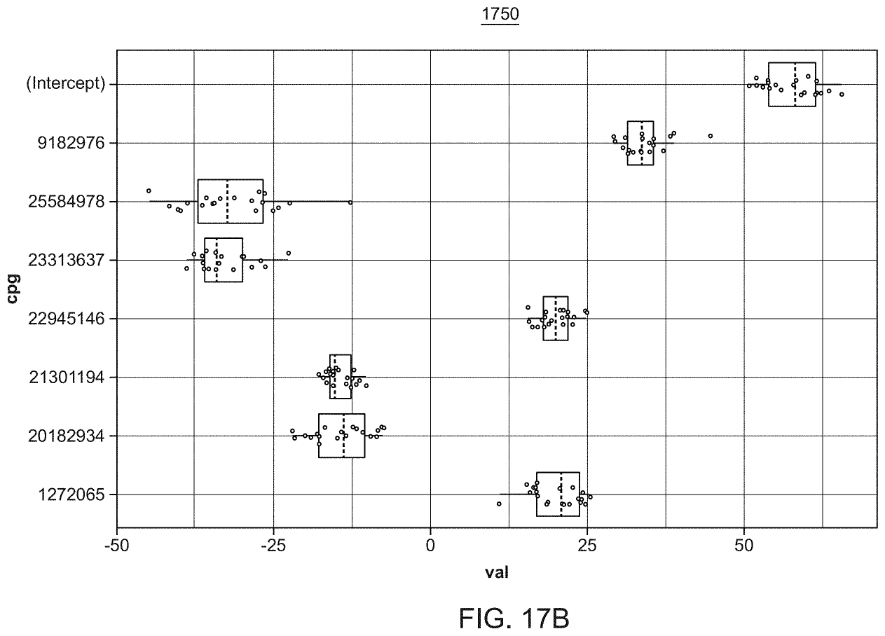

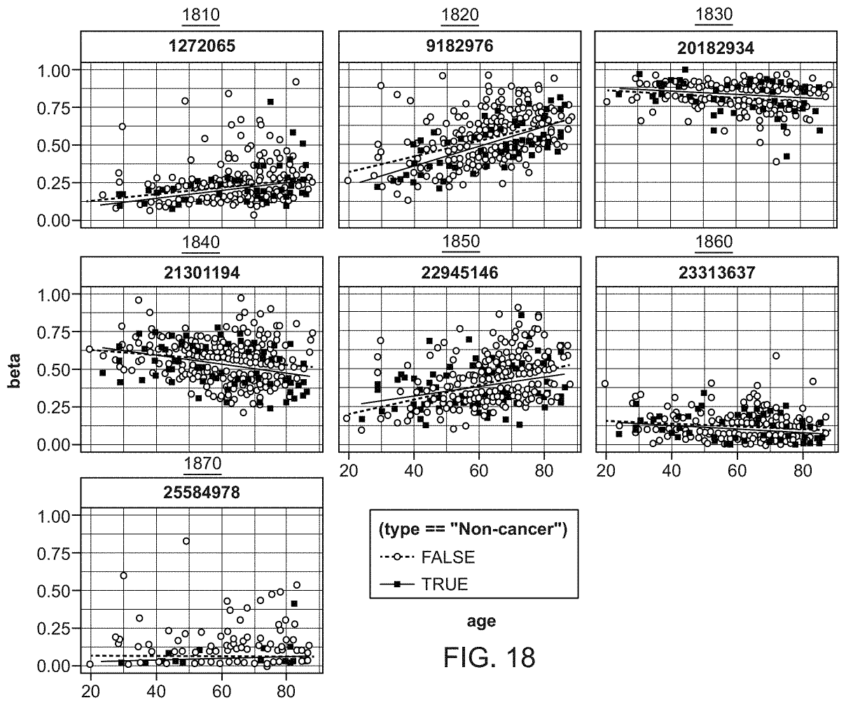

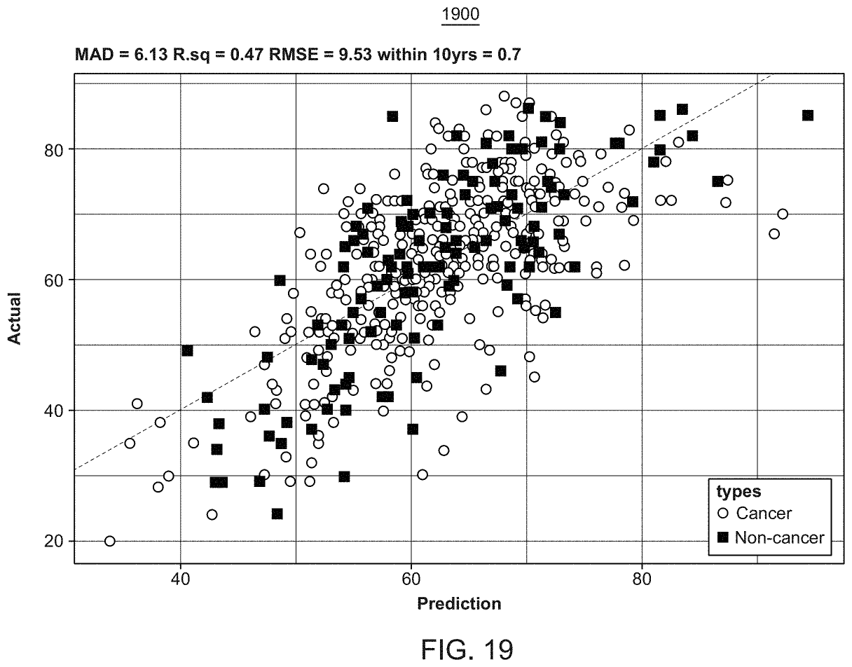

FIGS. 10-21 illustrate many graphs showing various characteristic prediction accuracy for use in sample swap validation. FIGS. 10 and 11 relate to biological sex prediction accuracy, according to principles described above in Section II.B.i. Biological Sex Prediction. FIGS. 12-16 relate to ethnicity prediction accuracy, according to principles described above in Section II.B.ii. Ethnicity Prediction. FIGS. 17A, 17B, and 18 relate to feature selection of informative CpG sites used in age prediction, and FIGS. 19-21 relate to age prediction accuracy, according to principles described above in Section II.B.iii. Age Prediction.

FIGS. 10 and 11 illustrate graphs depicting biological sex prediction accuracy. Graph 1000 in FIG. 10 illustrates biological sex prediction accuracy with samples from the CCGA study. For the samples used in the validation, the analytics system performed the process 320 for biological sex prediction in FIG. 3 using a threshold Y chromosome signal for predicting between biological male and biological female. The analytics system charted samples according to the calculated X chromosome signal and the calculated Y chromosome signal. As shown, samples in black (generally having values that plot in the top left of the graph 1000) were known to be biological male and were also accurately predicted to be biological male. Similarly, samples in white (generally having values that plot in the bottom right of the graph 1000) were known to biological female and were also accurately predicted to be biological female. Samples shown with diagonal lines were determined as having some level of contamination, with relative levels of contamination distinguished by the size of the circle representing the contaminated sample. Apart from the samples determined to have some level of contamination, the analytics system accurately predicted the test samples with 100% accuracy. Of note, the analytics system was still able to accurately predict four samples with sex chromosomal abnormalities. One sample with Turner Syndrome 1010 (having one X chromosome and a partial or missing X chromosome) was accurately predicted as biological female. One sample 1020 with Klinefelter Syndrome (having one Y chromosome and two X chromosomes) was accurately predicted as biological male. One sample 1030 having trisomy X (having three X chromosomes) was accurately predicted as biological female. One sample 1040 having tetrasomy X (having four X chromosomes) was accurately predicted as biological female.

Graph 1100 in FIG. 11 illustrates biological sex prediction accuracy with samples from the Compass Dev E2E study. With the samples used in the validation, the analytics system performed the process 320 in FIG. 3. To predict between biological female and biological male, the analytics system uses a Y chromosome threshold signal. The samples are plotted on the graph 1100 according to their X chromosome signal and their Y chromosome signal. Samples represented as black dots (generally having values that plot in the top left of the graph 1100) were known to be biological male and were also accurately predicted to be biological male. Samples represented as white dots (generally having values that plot in the bottom right of the graph 1100) were known to be biological female and were also accurately predicted to be biological female. Triangles represent samples that were determined to have some threshold level of contamination. Apart from the samples determined to have the threshold level of contamination, the biological prediction accuracy was 100%.

FIGS. 12-14 illustrate tables depicting ethnicity prediction accuracy across chromosomes. The plurality of SNPs considered in the ethnicity prediction for the samples depicted in FIGS. 12-14 were identified from the 1000 Genomes Project (also referred to as “1000G project”). Samples were classified to be from the following ethnicities as used by 1000G project: African, Admixed American, East Asian, European, and South Asian. The samples that were used to validate ethnicity prediction accuracy were chosen from the CCGA study. The CCGA study, however, requested reporting ethnicity to be one or more of: American Indian or Alaska Native; Asian, Native Hawaiian, or Pacific Islander; Black, non-Hispanic; White, non-Hispanic; and Hispanic. Between the two different sets of ethnicities, each ethnicity label used in CCGA reporting mapped best to the ethnicity label of 1000G project as follows: American Indian or Alaska Native mapped to Admixed American; Asian, Native Hawaiian, or Pacific Islander mapped to either East Asian or South Asian; Black, non-Hispanic mapped to African; White, non-Hispanic mapped to European; and Hispanic mapped to Admixed American. Despite this best mapping between the two different sets of ethnicities, some samples of one reported ethnicity may truly be of one or more of the ethnicity labels predicted for. The analytics system performed the process 325 in FIG. 5. This yielded an ethnicity probability for each autosomal chromosome, i.e., Chromosome 1 through Chromosome 22, for each ethnicity classified against. The analytics system further ranked the ethnicity predictions for each chromosome based on the calculated ethnicity probabilities.

The first sample shown in table 1200 was reported to be of white, non-Hispanic ethnicity, which best mapped to the European label. For the first sample, all chromosomes were in consensus having a first prediction of European. As a result, the analytics system returns the ethnicity prediction for the first sample of European, which was accurate for the reported white, non-Hispanic ethnicity label.

For the second sample shown in table 1300, the second sample was reported to be Asian, Native Hawaiian, or Pacific Islander, mapping to either East Asian or South Asian. All chromosomes were in consensus having a first prediction of East Asian. As a result, the analytics system returns the ethnicity prediction for the second sample of East Asian, which was accurate for the reported Asian, Native Hawaiian, or Pacific Islander ethnicity label.

The third sample, shown in table 1400, was reported to be of mixed ethnicity with Hispanic as the dominant ethnicity, Hispanic mapping best to Admixed American. Fourteen of the chromosomes had a first prediction of Admixed American with the remaining eight chromosomes having a first prediction of European. As a result, the analytics system returns a first ethnicity prediction of Admixed American with 14 chromosomes in support of the first prediction and a second ethnicity prediction of European with 8 chromosomes in support of the second prediction. Had the fourth sample returned a first prediction of European and a second prediction of Admixed American, then the analytics system would have still validated that the sample matched the reported ethnicity (as the second prediction matched the reported ethnicity). As with the fourth sample, returning first and second predictions aims to ensure samples of mixed ethnicities are not falsely invalidated.

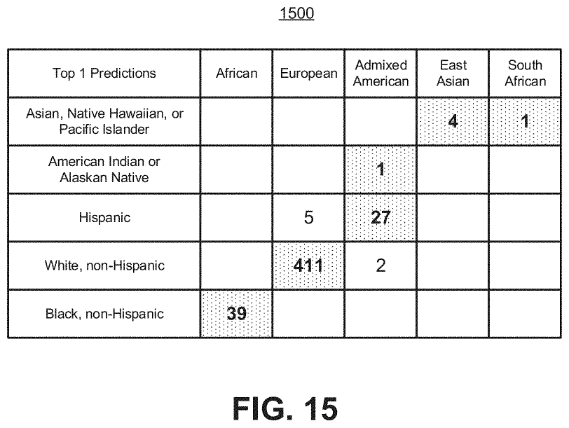

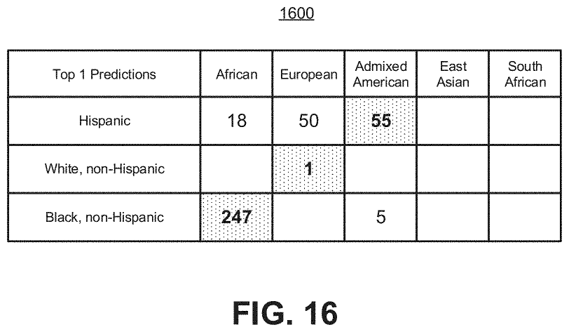

FIGS. 15 and 16 illustrate confusion matrices depicting ethnicity prediction accuracy with different sets of ethnicities used for classification. The reported ethnicity labels were the same as those used above in the results shown in FIGS. 12-14, used in the CCGA study. The ethnicity labels classified against were the same as those used above in the results shown in FIGS. 12-14, used in the 1000G project. The results of FIGS. 15 and 16 were achieved through the process 325 of FIG. 5.

Graph 1500 demonstrates robustness of the ethnicity prediction to cancer status. To achieve the results of graph 1500, the analytics system tested a set of 490 samples with 365 cancer samples and 125 non-cancer samples. In evaluating prediction accuracy, the analytics system utilized the top one prediction from the process 325 in FIG. 1. Samples reported to be of the ethnicity label of Asian, Native Hawaiian, or Pacific Islander were predicted to be of the ethnicity labels of East Asian or South Asian, as expected. One sample reported to be of the ethnicity label of American Indian or Alaska Native was predicted to be of the ethnicity label of Admixed American, as expected. Out of 32 samples reported to be of the ethnicity label of Hispanic, 27 samples predicted to be of the ethnicity label of Admixed American, as expected, but 5 were predicted to be of the ethnicity label of European which deviated from expectations. Out of 413 samples reported to be of the ethnicity label of White, non-Hispanic, 411 samples were predicted to be of the ethnicity label of European, as expected, but 2 samples were predicted to be of the ethnicity label of Admixed American which deviated from expectations. Samples reported to be of the ethnicity label of Black, non-Hispanic were predicted to be of the ethnicity label of African, as expected.

Graph 1600 demonstrates robustness in differing assays and varying SNP data available in each sample. To achieve the results of graph 1600, the analytics system tested a set of 376 samples from 56 individuals. From each individual, anywhere from one to sixteen samples were collected. The samples were assayed according to a plurality of assay protocols, yielding differential SNP data available in each sample. In evaluating prediction accuracy, the analytics system utilized the top one prediction from the process 325 in FIG. 1. Out of 123 samples reported to be of the ethnicity label of Hispanic, 18 were predicted to be the ethnicity label of African, 50 were predicted to be of the ethnicity label of white, non-Hispanic, and 55 were predicted to be of the ethnicity label of Admixed American. Although the Hispanic ethnicity label used the CCGA study best mapped to the Admixed American ethnicity label of the 1000G project, as in the case with these results, samples of the Hispanic ethnicity label used in the CCGA study had a widespread distribution of predictions. This could be due to the imprecise mapping between the two sets of ethnicity labels or simply due to Hispanic generally convoluted with other ethnicities. To circumvent false invalidations of samples with mixed or convoluted ethnicities, the analytics system may return top two ethnicity predictions in comparison with the reported ethnicity characteristic.

FIGS. 17A & 17B illustrates graphs depicting performance of features for feature selection. The analytics system retrieved information on 44 CpG sites known to be correlated with age from various studies. The analytics system took 20 sets of training samples to regress age in 20 different regression models. The learned coefficients from the 20 models are plotted in the graph 1700 in FIG. 17A. Each training set included around 500 or so samples. The graph 1750 in FIG. 17B identifies 7 of the more informative CpG sites which have the highest ratio of absolute mean over variance. From these 7 most informative CpG sites, the analytics system may evaluate the age prediction accuracy of regression models trained with different combination of features.

TABLE A
44 CpG Sites

1272065
1461244
3340188
3788543
3841767
3841769
4579774
4824592
5415579
7269214
7621451
8636528
8638735
9182976
9970373
12047610
12540957
13955257
14452305
15813350
16521727
17621034
17743159
17767122
18264541
18446434
18764748
20182934
20380293
20888124
21026889
21064282
21301194
21824804
22889160
22945146
23313637
24651959
24858581
24951611
25043027
25584978
26010281
26188974

FIG. 18 illustrates graphs depicting age prediction accuracy of each feature individually. The top 7 features were identified from the process described in FIGS. 17A & 17B. The top 7 CpG sites include CpG Site 1272065 shown in graph 1810, CpG Site 9182976 shown in graph 1820, CpG Site 20182934 shown in graph 1830, CpG Site 21301194 shown in graph 1840, CpG Site 22945146 shown in graph 1850, CpG Site 23313637 shown in graph 1860, and CpG Site 25584978 shown in graph 1870. Each of the graphs shows correlation between age on the x-axis and methylation density at the CpG site on the y-axis for a training set of training samples. Each graph also marks training samples that are non-cancer as blue and training samples that are cancer as red. All graphs show a strong correlation that is consistent between the non-cancer training samples and the cancer training samples.

FIG. 19 illustrates a graph 1900 depicting correlation between chronological age and determined age. The analytics system trains a linear regression model to predict age with a training set of non-cancer samples and cancer samples. The 44 features known to be correlated to age from various studies were used in training this example model. The analytics system validates the trained linear regression model yielding a median absolute deviation of 6.13, a R-squared of 0.47, a Root Mean Square Error (RMSE) of 9.53, and prediction accuracy within 10 years of 0.7.

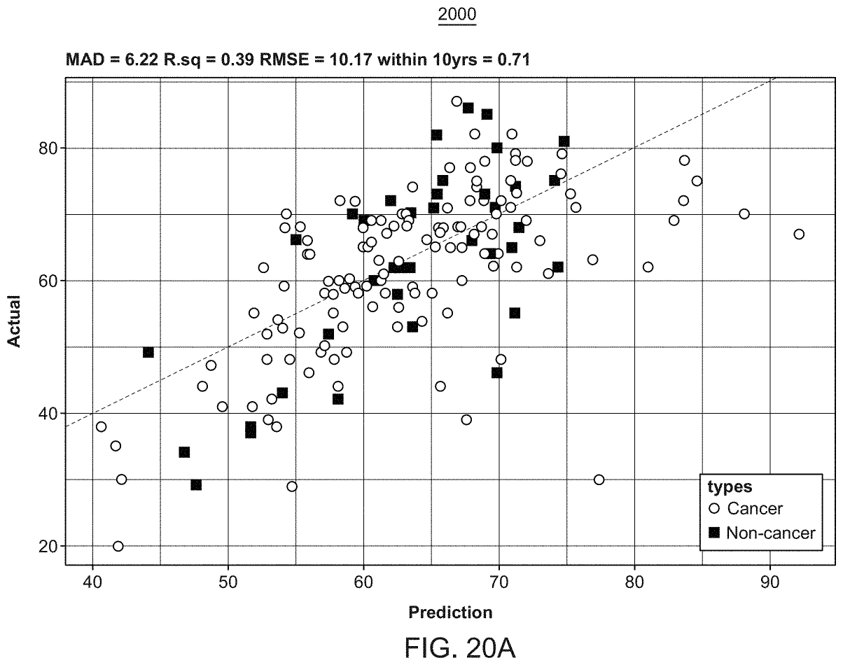

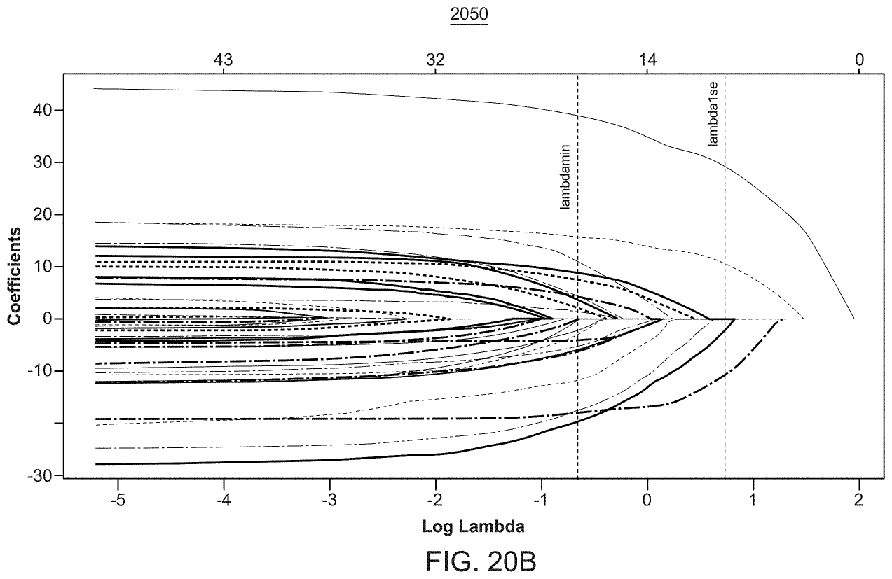

FIG. 20A illustrates a graph 2000 depicting age prediction accuracy with selected features and regularized performance. The analytics system implements a regularization factor from Glmnet's regression with regularization implementation. The analytics system validates the trained regression model with regularization yielding a median absolute deviation of 6.22, a R-squared of 0.39, a RMSE of 10.17, and prediction accuracy within 10 years of 0.71. FIG. 20B, graph 2050, illustrates the Glmnet's regularization of the variables in the regression.

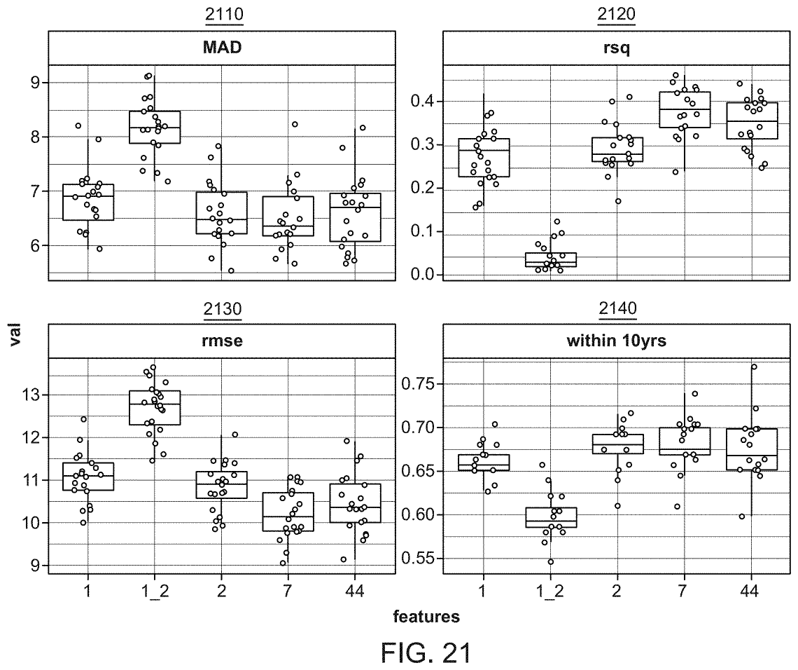

FIG. 21 illustrates graphs comparing age prediction accuracy considering different sets of features. Five different sets of features were used for age prediction to demonstrate the predictive accuracy between the different sets. A first set only considered the top 1^{st} feature determined in FIGS. 17A & 17B. A second set only considered the top 2^{nd} feature determined in FIGS. 17A & 17B. A third set considered the top 1^{st} and 2^{nd} features determined in FIG. 17 FIGS. 17A & 17B. A fourth set considered top 7 features determined in FIGS. 17A & 17B. A fifth set considered 44 features retrieved by the analytics system in FIGS. 17A & 17B. A regression model was trained with each set of features. Each trained regression model was validated with numerous test sets of samples. Various metrics for each trained regression model were evaluated and plotted. The first graph 2110 shows median absolute deviation. The second graph 2120 shows R-Squared. The third graph 2130 shows RMSE. The fourth graph 2140 shows prediction accuracy within 10 years of the true age. Notably, the regression model trained to consider the second set performed significantly worse than the others trained with other sets of features. The remaining sets performed similarly; however, the regression trained with the fourth set (inclusive of the top 7 features) performed slightly better with a higher R-Squared and a lower RMSE than the others. These graphs indicate

### VI. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method for validating that a cell-free deoxyribonucleic acid (cfDNA) sample is from a test subject, the method comprising:
obtaining a test sample from a test subject, wherein a biological sex of the test subject is known to be one of biological male or biological female;
obtaining the cfDNA sample from the test sample;
obtaining sequence reads from the cfDNA sample;
determining a first count of sequence reads for a first gene found on the Y chromosome and not found on the X chromosome;
normalizing the first count;
determining a Y chromosome signal for the cfDNA sample based on the normalized first count of sequence reads for the second gene;
determining a biological sex for the cfDNA sample based on the Y chromosome signal; and
validating that the cfDNA sample is from the test subject if the determined biological sex and the known biological sex are the same.

2. The method of claim 1, further comprising:
determining a second count of sequence reads for a second gene found on an X chromosome of the human genome and not found on a Y chromosome of the human genome;
normalizing the second count; and
determining an X chromosome signal for the cfDNA sample based on the normalized second count of sequence reads for the first gene;
wherein determining the biological sex for the cfDNA sample is further based on the X chromosome signal.

3. The method of claim 2, wherein the first count and the second count are normalized according to a sequencing depth of the cfDNA sample.

4. The method of claim 2, wherein determining the biological sex of the cfDNA sample comprises:
comparing a threshold ratio to a ratio of the Y chromosome signal for the cfDNA sample to the X chromosome signal for the cfDNA sample.

5. The method of claim 2, wherein determining the biological sex of the cfDNA sample comprises:
applying a biological sex classifier to the X chromosome signal for the cfDNA sample and the Y chromosome signal for the cfDNA sample to predict the biological sex of the cfDNA sample, wherein the biological sex classifier is trained with a training set of training samples, each training sample has a biological sex known to be one of biological male or biological female.

6. The method of claim 2, further comprising:
determining a third count of sequence reads for a third gene found on the Y chromosome and not found on the X chromosome;
determining a fourth count of sequence reads for a fourth gene found on the X chromosome and not found on the Y chromosome;
normalizing the third count and the fourth count;
wherein determining the Y chromosome signal is further based on the normalized third count; and
wherein determining the X chromosome signal is further based on the normalized fourth count.

7. The method of claim 6, wherein the first count, the second count, the third count, and the fourth count are normalized according to a sequencing depth of the cfDNA sample.

8. The method of claim 6, wherein the Y chromosome signal is an average of the normalized first count and the normalized third count, and wherein the X chromosome signal is an average of the normalized second count and the normalized fourth count.

9. The method of claim 1, wherein determining the biological sex of the cfDNA sample comprises:
comparing the Y chromosome signal for the cfDNA sample to a threshold Y chromosome signal,
wherein the cfDNA sample is determined to be biological male if the Y chromosome signal for the cfDNA sample is above the threshold Y chromosome signal, and
wherein the cfDNA sample is determined to be biological female if the Y chromosome signal for the cfDNA sample is below the threshold Y chromosome signal.

10. The method of claim 1, further comprising, responsive to validating the cfDNA sample:
filtering the sequence reads with p-value filtering to generate a set of anomalous fragments;
generating a test feature vector by generating, for each of a plurality of CpG sites, a score based on whether one or more anomalous fragments overlaps the CpG site;
inputting the test feature vector into a trained model to generate a cancer prediction for the test sample; and
determining whether the test sample is likely to have cancer according to the cancer prediction.

11. The method of claim 1, wherein the sequence reads comprise methylation sequencing data generated by methylation sequencing of the cfDNA fragments.

12. The method of claim 11, wherein the methylation sequencing comprises WGBS.

13. The method of claim 11, wherein the methylation sequencing comprises targeted sequencing.

14. A system comprising a hardware processor and a non-transitory computer-readable storage medium storing executable instructions that, when executed by the hardware processor, cause the processor to perform operations comprising the method of any of claims 1-13.

15. A method for validating that a cell-free deoxyribonucleic acid (cfDNA) sample is from a test subject, the method comprising:
obtaining a test sample from a test subject, wherein the test sample is reported to be one or more reported ethnicities of a plurality of ethnicities;
obtaining the cfDNA sample from the test subject;
obtaining a plurality of sequence reads from the cfDNA sample, the plurality of sequence reads including a plurality of single nucleotide polymorphisms (SNPs);
determining from the plurality of sequence reads, an allele frequency for each of the plurality of SNPs;
obtaining expected allele frequencies for each of the plurality of SNPs for each of the plurality of ethnicities determined from a training set, wherein the ethnicity is known for each training sample in the training set;
for each chromosome of a plurality of chromosomes:
calculating an ethnicity probability for each of the plurality of ethnicities based on the determined allele frequencies for a subset of SNPs within the chromosome and the expected allele frequencies for the plurality of ethnicities for the subset of SNPs within the chromosome;
predicting one or more ethnicities for the cfDNA sample based on the calculated ethnicity probabilities for the plurality of chromosomes; and
validating that the cfDNA sample is from the test subject based on the one or more predicted ethnicities of the cfDNA sample and the one or more reported ethnicities of the test subject.

16. The method of claim 15, further comprising:
determining a genotype for each of the plurality of SNPs based on the allele frequency at the SNP.

17. The method of claim 16, wherein, for each chromosome of the plurality of chromosomes, calculating the ethnicity probability for each of the plurality of ethnicities is further based on the determined genotypes for the subset of SNPs within the chromosome.

18. The method of claim 17, wherein, for each chromosome of the plurality of chromosomes, calculating the ethnicity probability for each of the plurality of ethnicities comprises calculating a Bayesian probability based on the determined genotypes for the subset of SNPs within the chromosome.

19. The method of claim 18, further comprising:
determining a genotype proportion of each ethnicity of the plurality of ethnicities for the determined genotype for each of the plurality of SNPs based on the expected allele frequencies for the plurality of ethnicities,
wherein calculating the Bayesian probability is further based on the determined genotype proportions.

20. The method of claim 15, further comprising:
for each chromosome of the plurality of chromosomes, ranking the plurality of ethnicities according to the determined ethnicity probabilities,
wherein a first predicted ethnicity comprises an ethnicity of the plurality of ethnicities corresponding to a largest number of the chromosomes ranking the first ethnicity first,
wherein a second predicted ethnicity comprises an ethnicity of the plurality of ethnicities corresponding to a second largest number of the chromosomes ranking the second ethnicity first, and
wherein validating that the cfDNA sample is from the test subject comprises determining that at least one of the first ethnicity prediction and the second ethnicity prediction matches one of the one or more reported ethnicities.

21. The method of claim 20, wherein a second predicted ethnicity comprises an ethnicity of the plurality of ethnicities corresponding to a second largest number of the chromosomes ranking the second ethnicity first.

22. The method of claim 21, wherein validating that the cfDNA sample is from the test subject comprises determining that at least one of the first ethnicity prediction and the second ethnicity prediction matches one of the one or more reported ethnicities.

23. The method of claim 15, further comprising, responsive to validating the cfDNA sample:
filtering the sequence reads with p-value filtering to generate a set of anomalous fragments;
generating a test feature vector by generating, for each of a plurality of CpG sites, a score based on whether one or more anomalous fragments overlaps the CpG site;
inputting the test feature vector into a trained model to generate a cancer prediction for the test sample; and
determining whether the test sample is likely to have cancer according to the cancer prediction.

24. The method of claim 15, wherein the sequence reads comprise methylation sequencing data generated by methylation sequencing of the cfDNA fragments.

25. The method of claim 24, wherein the methylation sequencing comprises WGBS.

26. The method of claim 24, wherein the methylation sequencing comprises targeted sequencing.

27. A system comprising a hardware processor and a non-transitory computer-readable storage medium storing executable instructions that, when executed by the hardware processor, cause the processor to perform operations comprising the method of any of claims 15-26.

28. A method for validating that a cell-free deoxyribonucleic acid (cfDNA) sample is from a test subject, the method comprising:
obtaining a test sample from a test subject, wherein an age of the test subject is reported to be within one of a plurality of age ranges;
receiving the cfDNA sample from the test sample;
obtaining sequence reads from the cfDNA sample;
for each of a plurality of CpG sites, determining a methylation density at each of the plurality of CpG sites based on the sequence reads from the cfDNA sample;
predicting an age range for the cfDNA sample by applying a trained regression model to the determined methylation densities for the plurality of CpG sites, wherein the trained regression model is trained using a training set where the methylation density for each of the plurality of CpG sites and an age is known for each individual of the training set;
validating that the cfDNA sample is from the test subject based on the predicted age range of the cfDNA sample and the reported age range of the test subject.

29. The method of claim 28, wherein the plurality of CpG sites is identified from an initial set of CpG sites found to be correlated with age, and wherein the plurality of CpG sites are identified by excluding CpG sites from the initial set of CpG sites that are confounding features for cancer prediction.

30. The method of claim 29, wherein the plurality of CpG sites is identified by further excluding CpG sites from the initial set of CpG sites that are confounding features for one or both of: biological sex and ethnicity.

31. The method of claim 28, wherein the plurality of CpG sites is identified by:
training a plurality of regression models, each regression model trained with a training set of training samples and comprising a learned coefficient for each CpG site of an initial set of CpG sites, wherein a learned coefficient for a given CpG site represents a predictive power of the CpG site;
for each CpG site of the initial set of CpG sites, determining an informative score calculated as an average of the learned coefficients for the CpG site over the plurality of regression models divided by a variance of the learned coefficients for the CpG site over the plurality of regression models;
ranking the CpG sites of the initial set of CpG sites according to the determined informative scores; and
selecting the plurality of CpG sites from the ranking.

32. The method of claim 28, wherein the trained regression model is trained using one of:
a linear regression operation
a logistic regression operation, and
a Glmnet's regression operation with regularization implementation.

33. The method of claim 28, wherein the trained regression model is trained using a logistic regression operation.

34. The method of claim 28, wherein the trained regression model is trained using a Glmnet's regression operation with regularization implementation

35. The method of claim 28, further comprising, responsive to validating the cfDNA sample:
filtering the sequence reads with p-value filtering to generate a set of anomalous fragments;
generating a test feature vector by generating, for each of a second plurality of CpG sites, a score based on whether one or more anomalous fragments overlaps the CpG site;
inputting the test feature vector into a trained model to generate a cancer prediction for the test sample; and
determining whether the test sample is likely to have cancer according to the cancer prediction.

36. The method of claim 28, wherein the sequence reads comprise methylation sequencing data generated by methylation sequencing of the cfDNA fragments.

37. The method of claim 36, wherein the methylation sequencing comprises WGB S.

38. The method of claim 36, wherein the methylation sequencing comprises targeted sequencing.

39. The method of claim 28, wherein the plurality of CpG sites comprise CpG sites listed in Table A.

40. A system comprising a hardware processor and a non-transitory computer-readable storage medium storing executable instructions that, when executed by the hardware processor, cause the processor to perform operations comprising the method of any of claims 27-39.

41. A method for validating that a cell-free deoxyribonucleic acid (cfDNA) sample is from a test subject, the method comprising:
obtaining a test sample from a test subject, wherein two or more of a biological sex, an ethnicity, and an age within one of a plurality of age ranges have been reported for the test subject;
obtaining the cfDNA sample from the test sample;
obtaining a plurality of sequence reads from the cfDNA sample;
predicting for the cfDNA sample two or more of:
a biological sex for the cfDNA sample based on:
a first count of sequence reads for a first gene found on an X chromosome of the human genome and not found on a Y chromosome of the human genome, and
a second count of sequence reads for a second gene found on the Y chromosome and not found on the X chromosome;
one or more ethnicities for the cfDNA sample based on ethnicity probabilities calculated for each chromosome of a plurality of chromosomes, the ethnicity probabilities for a given chromosome based on an allele frequency determined from the sequence reads of the cfDNA sample for each of a plurality of SNPs on the given chromosome; and
an age range for the cfDNA sample based on a methylation density determined for each of a plurality of CpG sites; and
validating that the cfDNA sample is from the test subject based on a comparison of two or more of the predicted biological sex of the cfDNA sample, the one or more predicted ethnicities of the cfDNA sample, the predicted age range of the cfDNA sample and two or more of the reported biological sex, the reported ethnicity, and the reported age range of the test subject.

42. A system comprising a hardware processor and a non-transitory computer-readable storage medium storing executable instructions that, when executed by the hardware processor, cause the processor to perform operations comprising the method of claim 41.

43. A method for validating that a cell-free deoxyribonucleic acid (cfDNA) sample is from a test subject, the method comprising:
obtaining a test sample from a test subject, wherein a biological sex and an ethnicity have been reported for the test subject;
obtaining the cfDNA sample from the test sample;
obtaining a plurality of sequence reads from the cfDNA sample;
predicting for the cfDNA sample:
a biological sex for the cfDNA sample based on:
a first count of sequence reads for a first gene found on an X chromosome of the human genome and not found on a Y chromosome of the human genome, and
a second count of sequence reads for a second gene found on the Y chromosome and not found on the X chromosome; and
one or more ethnicities for the cfDNA sample based on ethnicity probabilities calculated for each chromosome of a plurality of chromosomes, the ethnicity probabilities for a given chromosome based on an allele frequency determined from the sequence reads of the cfDNA sample for each of a plurality of SNPs on the given chromosome; and
validating that the cfDNA sample is from the test subject based on a comparison of the predicted biological sex of the cfDNA sample and the one or more predicted ethnicities of the cfDNA sample to the reported biological sex and the reported ethnicity of the test subject.

44. A system comprising a hardware processor and a non-transitory computer-readable storage medium storing executable instructions that, when executed by the hardware processor, cause the processor to perform operations comprising the method of claim 43.

45. A method for validating that a cell-free deoxyribonucleic acid (cfDNA) sample is from a test subject, the method comprising:
obtaining a test sample from a test subject, wherein a biological sex and an age within one of a plurality of age ranges have been reported for the test subject;
obtaining the cfDNA sample from the test sample;
obtaining a plurality of sequence reads from the cfDNA sample;
predicting for the cfDNA sample:
a biological sex for the cfDNA sample based on:
a first count of sequence reads for a first gene found on an X chromosome of the human genome and not found on a Y chromosome of the human genome, and
a second count of sequence reads for a second gene found on the Y chromosome and not found on the X chromosome; and
an age range for the cfDNA sample based on a methylation density determined for each of a plurality of CpG sites; and
validating that the cfDNA sample is from the test subject based on a comparison of the predicted biological sex of the cfDNA sample and the predicted age range of the cfDNA sample to the reported biological sex and the reported age range of the test subject.

46. A system comprising a hardware processor and a non-transitory computer-readable storage medium storing executable instructions that, when executed by the hardware processor, cause the processor to perform operations comprising the method of claim

45.

47. A method for validating that a cell-free deoxyribonucleic acid (cfDNA) sample is from a test subject, the method comprising:
obtaining a test sample from a test subject, wherein an ethnicity and an age within one of a plurality of age ranges have been reported for the test subject;
obtaining the cfDNA sample from the test sample;
obtaining a plurality of sequence reads from the cfDNA sample;
predicting for the cfDNA sample:
one or more ethnicities for the cfDNA sample based on ethnicity probabilities calculated for each chromosome of a plurality of chromosomes, the ethnicity probabilities for a given chromosome based on an allele frequency determined from the sequence reads of the cfDNA sample for each of a plurality of SNPs on the given chromosome; and
an age range for the cfDNA sample based on a methylation density determined for each of a plurality of CpG sites; and
validating that the cfDNA sample is from the test subject based on a comparison of the one or more predicted ethnicities of the cfDNA sample and the predicted age range of the cfDNA sample to the reported ethnicity and the reported age range of the test subject.

48. A system comprising a hardware processor and a non-transitory computer-readable storage medium storing executable instructions that, when executed by the hardware processor, cause the processor to perform operations comprising the method of claim 47.
